(19) United States
(12) Patent Application Publication
(10) Pub. No.: US 2006/0014673 A1
(43) Pub. Date: Jan. 19, 2006

(54) Kappa-A conopeptides and uses therefor

(75) Inventors: Richard T. Layer, Sandy, UT (US); Karen E. Pemberton, Guilford, CT (US); Robert M. Jones, Park City, UT (US); James L. Garrett, Salt Lake City, UT (US); Baldomero M. Olivera, Salt Lake City, UT (US); J. Michael McIntosh, Salt Lake City, UT (US); David R. Hillyard, Salt Lake City, UT (US); Michelle Grilley, Logan, UT (US); Maren Watkins, Salt Lake City, UT (US); Ameurfina D. Santos, Quezon City (PH); Glenn Zafaralla, Livonia, MI (US); A. Grey Craig, Solana Beach, CA (US)

Correspondence Address: ROTHWELL, FIGG, ERNST & MANBECK, P.C., 1425 K STREET, N.W., SUITE 800, WASHINGTON, DC, 20005, US

(73) Assignees: Cognetix, Inc., Salt Lake City, UT (US); University of Utah Research Foundation, Salt Lake City, UT (US)

(21) Appl. No.: 11/174,996

(22) Filed: Jul. 6, 2005

Related U.S. Application Data
(63) Continuation of application No. 10/139,272, filed on May 7, 2002, now abandoned.
Application No. 10/139,272 is a continuation of application No. 09/413,354, filed on Oct. 6, 1999, now abandoned.
(60) Provisional application No. 60/103,247, filed on Oct. 6, 1998.

(51) Int. Cl. A61K 38/16; C07K 14/435

(52) U.S. Cl. 514/8; 530/322; 530/324

(57) ABSTRACT

The present invention is directed to kappaA (κA) conopeptides and the use of these peptides for blocking the flow of potassium ions through voltage-gated potassium channels. The κA conopeptides include unglycosylated and O-glycosylated peptides.

### CROSS-REFERENCE TO RELATED APPLICATION

The present application is a continuation of U.S. patent application Ser. No. 10/139,272 filed on 7 May. 2002, which in turn is a continuation of U.S. patent application Ser. No. 09/413,354 filed on 6 Oct. 1999, which in turn is related to and claims priority under 35 USC §119(e) to U.S. provisional application Ser. No. 60/103,247, filed 6 Oct. 1998, each incorporated herein by reference.

This invention was made with Government support under Grant No. GM-48677 awarded by the National Institutes of Health, Bethesda, Maryland. The United States Government has certain rights in the invention.

### BACKGROUND OF THE INVENTION

The present invention is directed to kappaA (κA) conopeptides and the use of these peptides for blocking the flow of potassium ions through voltage-gated potassium channels. The κA conopeptides include unglycosylated and O-glycosylated peptides.

The publications and other materials used herein to illuminate the background of the invention, and in particular, cases to provide additional details respecting the practice, are incorporated by reference, and for convenience are numerically referenced in the following text and respectively grouped in the appended bibliography.

Mollusks of the genus Conus produce a venom that enables them to carry out their unique predatory lifestyle. Prey are immobilized by the venom that is injected by means of a highly specialized venom apparatus, a disposable hollow tooth that functions both in the manner of a harpoon and a hypodermic needle.

Few interactions between organisms are more striking than those between a venomous animal and its envenomated victim. Venom may be used as a primary weapon to capture prey or as a defense mechanism. Many of these venoms contain molecules directed to receptors and ion channels of neuromuscular systems.

The predatory cone snails (Conus) have developed a unique biological strategy. Their venom contains relatively small peptides that are targeted to various neuromuscular receptors and may be equivalent in their pharmacological diversity to the alkaloids of plants or secondary metabolites of microorganisms. Many of these peptides are among the smallest nucleic acid-encoded translation products having defined conformations, and as such, they are somewhat unusual. Peptides in this size range normally equilibrate among many conformations. Proteins having a fixed conformation are generally much larger.

The cone snails that produce these toxic peptides, which are generally referred to as conotoxins or conotoxin peptides, are a large genus of venomous gastropods comprising approximately 500 species. All cone snail species are predators that inject venom to capture prey, and the spectrum of animals that the genus as a whole can envenomate is broad. A wide variety of hunting strategies are used, however, every Conus species uses fundamentally the same basic pattern of envenomation.

Several peptides isolated from Conus venoms have been characterized. These include the α-, μ- and ω-conotoxins which target nicotinic acetylcholine receptors, muscle sodium channels, and neuronal calcium channels, respectively (Olivera et al., 1985). Conopressins, which are vasopressin analogs, have also been identified (Cruz et al.. 1987). In addition, peptides named conantokins have been isolated from Conus geographus and Conus tulipa (Mena et al., 1990; Haack et al., 1990). These peptides have unusual age-dependent physiological effects: they induce a sleep-like state in mice younger than two weeks and hyperactive behavior in mice older than 3 weeks (Haack et al., 1990). The isolation, structure and activity of κ-conotoxins (now named κA conotoxins) are described in U.S. Pat. No. 5,633,347. Recently, peptides named contryphans containing D-tryptophan residues have been isolated from Conus radiatus (U.S. Ser. No. 09/061,026), and bromo-tryptophan conopeptides have been isolated from Conus imperialis and Conus radiatus (U.S. Ser. No. 08/785,534).

Potassium channels comprise a large and diverse group of proteins that, through maintenance of the cellular membrane potential, are fundamental in normal biological function. These channels are vital in controlling the resting membrane potential in excitable cells and can be broadly sub-divided into three classes: voltage-gated K⁺ channels, Ca²⁺ activated K⁺ channels and ATP-sensitive K⁺ channels. Many disorders are associated with abnormal flow of potassium ions through these channels. The identification of agents which would regulate the flow of potassium ions through each of these channel types would be useful in treating disorders associated with such abnormal flow.

It is desired to identify additional conotoxin peptides having activities of the above conopeptides, as well as conotoxin peptides having additional activities.

### SUMMARY OF THE INVENTION

The present invention is directed to kappaA (κA) conopeptides and the use of these peptides for blocking the flow of potassium ions through voltage-gated potassium channels. The κA conopeptides described herein are useful for treating various disorders as described in further detail herein. The κA conopeptides include unglycosylated and O-glycosylated peptides.

In one embodiment, the present invention is directed to κA conopeptides, κA conopeptide propeptides and nucleic acids encoding these peptides. The κA conopeptides have the following formulas:

κA A10.1: Xaa2-Lys-Xaa1-Leu-Val-Val-Thr-Ala-Thr-Thr-Thr-Cys-Cys-Gly-Xaa5-Asn-Xaa3-Met-Thr-Ser-Cys-Xaa3-Arg-Cys-Met-Cys-Asp-Ser-Ser-Cys-Xaa6 (SEQ ID NO: 1)

κA A10.2: Xaa2-Xaa3-Xaa4-Leu-Val-Xaa3-Ser-Lys-Ile-Thr-Asn-Cys-Cys-Gly-Xaa5-Asn-Asn-Met-Xaa1-Met-Cys-Xaa3-Thr-Cys-Met-Cys-Thr-Xaa5-Ser-Cys-Xaa7 (SEQ ID NO:2)

κA C10.1a: Xaa2-Lys-Xaa1-Leu-Val-Xaa3-Ser-Thr-Ile-Thr-Thr-Cys-Cys-Gly-Asn-Xaa1-Xaa3-Gly-Thr-Met-Cys-Xaa3-Lys-Cys-Met-Cys-Asp-Asn-Thr-Cys-Xaa8 (SEQ ID NO:3)

κA C10.1b: Xaa2-Lys-Xaa1-Leu-Val-Xaa3-Ser-Thr-Ile-Thr-Thr-Cys-Cys-Gly-His-Xaa1-Xaa3-Gly-Thr-Met-Cys-Xaa3-Lys-Cys-Met-Cys-Asp-Asn-Thr-Cys-Xaa8 (SEQ ID NO:4)

κA C10.2: Xaa2-Lys-Xaa1-Leu-Val-Val-Thr-Ala-Thr-Thr-Thr-Cys-Cys-Gly-Xaa5-Asn-Xaa3-Met-Ser-Met-Cys-Xaa3-Lys-Cys-Met-Cys-Thr-Xaa5-Ser-Cys-Xaa9 (SEQ ID NO:5)

κA Cr10.1: Xaa2-Xaa3-Xaa4-Leu-Val-Xaa3-Ser-Thr-Ile-Thr-Thr-Cys-Cys-Gly-Xaa5-Asp-Xaa3-Gly-Thr-Lys-Cys-Xaa3-Xaa3-Cys-Arg-Cys-Asn-Asn-Thr-Cys-Xaa10 (SEQ ID NO:6)

κA Cn10.1: Ala-Xaa3-Xaa4-Leu-Val-Xaa3-Ser-Gln-Ile-Thr-Thr-Cys-Cys-Gly-Xaa5-Asn-Xaa3-Gly-Thr-Met-Cys-Xaa3-Ser-Cys-Met-Cys-Thr-Asn-Ser-Cys (SEQ ID NO:7)

κA Cn10.2: Xaa2-Lys-Asp-Leu-Val-Val-Thr-Ala-Thr-Thr-Thr-Cys-Cys-Gly-Xaa5-Asn-Xaa3-Met-Thr-Ile-Cys-Xaa3-Xaa3-Cys-Met-Cys-Thr-Xaa5-Ser-Cys-Xaa11 (SEQ ID NO:8)

κA M10.2: Ala-Xaa3-Xaa1-Leu-Val-Val-Thr-Ala-Thr-Thr-Thr-Cys-Cys-Gly-Phe-Asp-Xaa3-Met-Thr-Xaa4-Cys-Xaa3-Xaa3-Cys-Met-Cys-Thr-Xaa5-Ser-Cys-Xaal2 (SEQ ID NO:9)

κA U006: Ala-Xaa3-Xaa1-Leu-Val-Val-Thr-Ala-Thr-Thr-Asn-Cys-Cys-Gly-Xaa5-Asn-Xaa3-Met-Thr-Ile-Cys-Xaa3-Xaa3-Cys-Met-Cys-Thr-Xaa5-Ser-Cys-Xaa13 (SEQ ID NO: 10)

κA Mn 10.1: Xaa2-Lys-Xaa1-Leu-Val-Val-Thr-Ala-Thr-Thr-Thr-Cys-Cys-Gly-Xaa5-Asn-Xaa3-Met-Thr-Ser-Cys-Xaa3-Arg-Cys-Met-Cys-Asp-Ser-Ser-Cys-Xaa6 (SEQ ID NO: 11)

κA Mn10.2: Xaa2-Xaa3-Xaa4-Leu-Val-Xaa3-Ser-Lys-Ile-Thr-Asn-Cys-Cys-Gly-Xaa5-Asn-Thr-Met-Xaal -Met-Cys-Xaa3-Thr-Cys-Met-Cys-Thr-Xaa5-Ser-Cys-Xaal 4 (SEQ ID NO: 12)

κcA Sm10.2: Ala-Xaa3-Xaa4-Leu-Val-Xaa3-Ser-Thr-Ile-Thr-Thr-Cys-Cys-G1y-Xaa5-Asp-Xaa3-Gly-Thr-Met-Cys-Xaa3-Xaa3-Cys-Met-Cys-Asn-Asn-Thr-Cys-Xaa15 (SEQ ID NO: 13)

κA Sm10.3: Xaa2-Ala-Xaa3-Leu-Val-Xaa3-Ser-Thr-lle-Thr-Thr-Cys-Cys-Gly-Xaa5-Asp-Xaa3-Gly-Thr-Met-Cys-Xaa3-Thr-Cys-Met-Cys-Asn-Asn-Thr-Cys-Xaa16 (SEQ ID NO: 14)

κA SmVIII: Xaa2-Thr-Xaa4-Leu-Val-Xaa3-Ser-Thr-Ile-Thr-Thr-Cys-Cys-Gly-Xaa5-Asp-Xaa3-Gly-Thr-Met-Cys-Xaa3-Thr-Cys-Met-Cys-Asp-Asn-Thr-Cys-Xaa16 (SEQ ID NO:1 5)

κA SmVIIIA: Ala-Xaa3-Xaa4-Leu-Val-Xaa3-Ser-Thr-Ile-Thr-Thr-Cys-Cys-Gly-Xaa5-Asp-Xaa3-Gly-Ser-Met-Cys-Xaa3-Xaa3-Cys-Met-Cys-Asn-Asn-Thr-Cys-Xaa17 (SEQ ID NO: 16)

δA SIVA: Xaa2-Lys-Ser-Leu-Val-Xaa3-Ser-Val-Ile-Thr-Thr-Cys-Cys-Gly-Xaa5-Asp-Xaa3-Gly-Thr-Met-Cys-Xaa3-Xaa3-Cys-Arg-Cys-Thr-Asn-Ser-Cys (SEQ ID NO: 17)

κA SVIIIA: Xaa2-Lys-Xaal-Leu-Val-Xaa3-Ser-Val-Ile-Thr-Thr-Cys-Cys-Gly-Xaa5-Asp-Xaa3-Gly-Thr-Met-Cys-Xaa3-Xaa3-Cys-Arg-Cys-Thr-Asn-Ser-Cys-Xaa18 (SEQ ID NO: 18)

κA Sx10.1: Xaa2-Ser-Xaa4-Leu-Val-Xaa3-Ser-Thr-Ile-Thr-Thr-Cys-Cys-Gly-Xaa5-Ser-Xaa3-Gly-Thr-Met-Cys-Xaa3-Xaa3-Cys-Met-Cys-Thr-Asn-Thr-Cys (SEQ ID NO: 19)

κA S110.1: Xaa2-Lys-Asp-Leu-Val-Val-Thr-Ala-Thr-Thr-Thr-Cys-Cys-Gly-Xaa5-Asn-Xaa3-Met-Thr-Met-Cys-Xaa3-Xaa3-Cys-Met-Arg-Thr-Xaa5-Ser-Cys-Xaal9 (SEQ ID NO:20)

κA S110.2: Xaa2-Lys-Xaa1-Leu-Val-Xaa3-Ser-Val-Ile-Thr-Thr-Cys-Cys-Gly-Xaa5-Asp-Xaa3-Gly-Thr-Met-Cys-Xaa3-Xaa3-Cys-Arg-Cys-Thr-Asn-Ser-Cys-Xaa18 (SEQ ID NO:21)

κA A671: Ala-Xaa3-Xaa4-Leu-Val-Xaa3-Ser-Thr-Ile-Thr-Thr-Cys-Cys-Gly-Xaa5-Asn-Xaa3-Gly-Thr-Met-Cys-Xaa3-Xaa3-Cys-Arg-Cys-Asp-Asn-Thr-Cys (SEQ ID NO:22)

κA H350: Xaa2-Ser-Xaa4-Leu-Val-Xaa3-Ser-Thr-Ile-Thr-Thr-Cys-Cys-Gly-Xaa5-Asp-Xaa3-Gly-Thr-Met-Cys-Xaa3-Xaa3-Cys-Arg-Cys-Asn-Asn-Thr-Cys-Xaa10 (SEQ ID NO:23)

κA J454: Ala-Xaa3-Xaa1-Leu-Val-Val-Thr-Ala-Thr-Thr-Thr-Cys-Cys-Gly-Xaa5-Asp-Xaa3-Met-Thr-Ile-Cys-Xaa3-Xaa3-Cys-Met-Cys-Thr-His-Ser-Cys-Xaa13 (SEQ ID NO:24)

κA G851: Ala-Xaa3-Xaa1-Leu-Val-Val-Thr-Ala-Thr-Thr-Thr-Cys-Cys-Gly-Xaa5-Asp-Xaa3-Met-Thr-Xaa4-Cys-Xaa3-Ser-Cys-Met-Cys-Thr-Xaa5-Ser-Cys-Xaa20 (SEQ ID NO:25),

wherein Xaa1 is Glu or γ-carboxy-Glu, Xaa2 is Gln or pyro-Glu, Xaa3 is Pro or hydroxy-Pro, Xaa4 is Trp, D-Trp or bromo-Trp, Xaa5 is Tyr, mono-iodo-Tyr, di-iodo-tyr, O-sulpho-Tyr, O-phospho-Tyr or nitro-Tyr, Xaa6 is des-Xaa6 or a peptide X1-Y1, Xaa7 is des-Xaa7 or a peptide X2-Y2, Xaa8 is des-Xaa8 or a peptide X3-Y1, Xaa9 is des-Xaa9 or a peptide X4-Y1, Xaa10 is des-Xaa10 or a peptide X5-Y3, Xaa11 is des-Xaa11 or a peptide X6-Y1, Xaa12 is des-Xaa12 or a peptideX7-Y1, Xaal3 is des-Xaa13 or a peptide X8-Y1, Xaa14 is des-Xaa14 or a peptide X2-Y1, Xaa15 is des-Xaa15 or a peptide X9-Y1, Xaa16 is des-Xaa16 or a peptide X10-Y1, Xaa17 is des-Xaa17 or a peptide X10-Y4, Xaa18 is des-Xaa18 or a peptide X11-Y1, Xaa19 is des-Xaa19 or a peptide X12-Y1, Xaa20 is des-Xaa20 or a peptide X12-Y1, X1 is Asn-Lys-Lys-Lys-Xaa3 (SEQ ID NO:26), X2 is Arg-Xaa3-Lys-Lys-Lys-Lys-Xaa3 (SEQ ID NO:27), X3 is Xaa3-Xaa3-Lys-Lys-Lys-Lys-Arg-Xaa3 (SEQ ID NO:28), X4 is Xaa3-His-Gln-Lys-Lys-Lys-Arg-Xaa3 (SEQ ID NO:29), X5 is Lys-Xaa3-Lys-Lys-Xaa3-Lys-Xaa3 (SEQ ID NO:30), X6 is Xaa3-Xaa3-Lys-Lys-Lys-Lys-Xaa3 (SEQ ID NO:31), X7 is Ser-His-Gln-Arg-Lys-Lys-Xaa3 (SEQ ID NO:32), X8 is Xaa3-Xaa3-Lys-Arg-Lys-Xaa3 (SEQ ID NO:33), X9 is Lys-Xaa3-Thr-Lys-Lys-Arg-Xaa3 (SEQ ID NO:34), X10 is Lys-Xaa3-Lys-Xaa3-Lys-Lys-Ser (SEQ ID NO:35), X11 is Xaa3-Thr-Lys-Xaa3-Lys-Lys-Xaa3 (SEQ ID NO:36), X12 is Ser-Xaa3-Lys-Lys-Lys-Lys-Xaa3 (SEQ ID NO:37), X13 is Xaa3-His-Gln-Arg-Lys-Lys-Xaa3 (SEQ ID NO:38), Y1 is Gly-Arg-Arg-Asn-Asp (SEQ ID NO:39), Y2 is Gly-His-Arg-Asn-Asp (SEQ ID NO:40), Y3 is Gly-Lys or Gly-Lys-Gly-Arg-Arg-Asn-Asp (SEQ ID NO:41), and Y4 is Gly-Arg-Arg-Asn-His (SEQ ID NO:42).

In a second embodiment, the present invention is directed to glycosylated κA conopeptides. These glycosylated κA conopeptides include the above κA conopeptides is which one or more of the hydroxylated residues have been modified to contain an O-glycan. It is preferred that the the amino acid in the seventh position contain an O-glycan. In accordance with the present invention, an O-glycan shall mean any S- or O-linked mono-, di-, tri-, poly- or oligosaccharide that can be attached to any hydroxy, amino or thiol group of natural or modified amino acids by synthetic or enzymatic methodologies known in the art. The monosaccharides making up the O-glycan can include D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified with one or more O-sulfate, O-phosphate or acidic groups, such as sialic acid, including combinations thereof. The gylcan may also include similar polyhydroxy groups, such as D-penicillamine 2,5 and halogenated derivatives thereof or polypropylene glycol derivatives. The glycosidic linkage is beta, preferably 1-3. The GalNAc-(aa) or GlcNAc-(aa) linkage is alpha and is 1-, wherein (aa) is the amino acid to which the glycan is attached. Preferred O-glycans are described further herein.

In a third embodiment, the present invention is directed to κA conopeptides having the following general formula,

Xaa1-Xaa2-Xaa3-Leu-Val-Xaa4-Xaa5-Xaa6-Xaa7-Thr-Thr-Cys-Cys-Gly-Xaa8-Xaa9-Xaa4-Xaa10-Xaa5-Xaa11-Cys-Xaa12-Xaa12-Cys-Xaa13-Cys-Xaa14-Xaa15-Xaa12-Cys-Xaa16 (SEQ ID NO:43),

wherein Xaa1 is Ala, Glu, Gln, pyro-Glu or γ-carboxy-Glu, Xaa2 is Pro, hydroxy-Pro, Ser, Thr or Lys, Xaa3 is Trp, D-Trp, bromo-Trp, Glu or γ-carboxy-Glu, Xaa4 is Pro, hydroxy-Pro or Val, Xaa5 is Ser or Thr, Xaa6 is Ala, Thr or Val, Xaa7 is Thr or Ile, Xaa8 is Tyr, mono-iodo-Tyr, di-iodo-Tyr, O-sulpho-Tyr, O-phospho-Tyr or nitro-Tyr, Xaa9 is Asp or Asn, Xaa10 is Met or Gly, Xaa11 is Met, Trp, D-Trp, bromo-Trp, Ile, Nle or Leu, Xaal2 is Pro, hydroxy-Pro, Ser or Thr, Xaa13 is Arg or Met, Xaa14 is Asp, Asn, Thr or Ser, Xaa15 is Asn, His or Tyr,mono-iodo-Tyr, di-iodo-Tyr, O-sulpho-Tyr, O-phospho-Tyr or nitro-Tyr and Xaa16 is des-Xaa16 or a peptide. The peptide has the formula A-B where A is peptide selected from the group of peptides having SEQ ID NOs:26-38 and B is des-B or a peptide selected from the group of peptides having SEQ ID NOs:39-42. The C-terminus contains a carboxyl group or is amidated. These peptides may further contain one or more O-glycans as described above. The O-glycans may occur at residues 7, 9, 10, 11, 19,27 and 29.

In a fourth embodiment, the present invention is directed to a consensus κA conopeptide having the formula,

Xaa1-Xaa2-Xaa3-Leu-Val-Xaa4-Ser-Xaa5-Ile-Thr-Thr-Cys-Cys-Gly-Tyr-Asp-Xaa4-Gly-Thr-Met-Cys-Xaa4-Xaa4-Cys-Xaa6-Cys-Thr-Asn-Xaa7-Cys (SEQ ID NO:44)

wherein Xaa1 is Ala, Glu, Gln, pyro-Glu or γ-carboxy-Glu, Xaa2 is Pro, hydroxy-Pro, Ser, Thr or Lys, Xaa3 is Trp, D-Trp, bromo-Trp, Glu or γ-carboxy-Glu, Xaa4 is Pro or hydroxy-Pro, Xaa5 is Ala, Thr or Val, Xaa6 is Met or Arg and Xaa7 is Thr or Ser. The C-terminus contains a free carboxyl group or is amidated. It is preferred that the C-terminus is amidated. These peptides may further contain one or more O-glycans as described above. The O-glycans may occur at residues 7,9,10,11, 19,27and 29.

In a fifth embodiment, the present invention is directed to uses of the κA conopeptides described herein for regulating the flow of potassium ions through K⁺ channels. Disorders which can be treated using these conopeptides include multiple sclerosis, other demyelinating diseases (such as acute dissenmiated encephalomyelitis, optic neuromyelitis, adrenoleukodystrophy, acute transverse myelitis, progressive multifocal leukoencephalopathy), sub-acute sclerosing panencephalomyelitis (SSPE), metachromatic leukodystrophy, Pelizaeus-Merzbacher disease, spinal cord injury, botulinum toxin poisoning, Huntington's chorea, compression and entrapment neurophathies (such as carpal tunnel syndrome, ulnar nerve palsy), cardiovascular disorders (such as cardiac arrhythmias, congestive heart failure), reactive gliosis, hyperglycemia, immunosuppression, cocaine addiction, cancer, cognitive dysfunction, disorders resulting from defects in neurotransmitter release (such as Eaton-Lambert syndrome), and reversal of the actions of curare and other neuromuscular blocking drugs.

### BRIEF DESCRIPTION OF THE FIGURES

FIGS. 1A and 1B show the native O-glycans which maybe attached to Ser7 of κA SIVA.

FIGS. 2A-2C show the native O-glycans which may be attached to Thr7 and Thr9 of κA U006.

FIG. 3 shows the preferred core O-glycans (Van de Steen et al., 1998). Mucin type O-linked oligosaccharides are attached to Ser or Thr (or other hydroxylated residues of the present peptides) by a GalNAc residue. The monosaccharide building blocks and the linkage attached to this first GalNAc residue define the “core glycans,” of which eight have bee identified. The type of glycosidic linkage (orientation and connectivities) are defined for each core glycan.

FIG. 4 shows κA A671 induces an increase in intracellular calcium in a depolarized environment. The data represents mean+SEM fluorescence collected from two to 10 individual trials. Cells were depolarized with 1-10 μM Aconitine. Data shown was collected at 15 min, compared to the aconitine pretreatment values and corrected to the response to Aconitine alone.

FIG. 5 shows response to κA A671 is sustained through time. Values shown represent mean+SEM responses to 1 μM κA A671 peaks 1 and 2. Data indicates % change in fluorescence from Aconitine pretreatment values (10 individual trials each). Data is also corrected to the Aconitine application alone through time.

FIGS. 6A-6C show the increase in intracellular calcium induced by κA A671 peak 2 is inhibited by pretreating of 4-AP. FIG. 6A shows the increase in intracellular calcium induced by 4-AP pretreatment and by a subsequent addition of κA A671 (2 μM) in the presence of the 4-AP. Experiments were carried out in the presence of aconitine (10 μM) or brevetoxin (100 nM), and were corrcted to the responses induced by the depolarizing agent alone. FIG. 6B shows an enlarged portion of FIG. 6A, to show the reduction in amplitude of the κA A671 response. Data shown in FIG. 6C is the response to κA A671 following subtraction of the 4-AP response, i.e., (response to κA A671+4-AP)—(response to 4-AP pretreatment) from the same cells (same data as in FIGS. 6A+6B). As with FIGS. 6A and 6B, the data is corrected to a parallel addition of aconitine. Values for all graphs shown represent the mean+SEM % change in fluorescence. All data is from the same 2-5 individual trials.

FIG. 7 shows that pretreatment with Dendrotoxin in the presence of aconitine has no effect on the κA A671 induced response. The graph shows the increase in intracellular calcium induced by exposure to Dendrotoxin (two individual trials). The graph also shows the effect of exposing to κA A671 peak 2 (2 μM) in cells that have been pretreated with aconitine and dendrotoxin. The values are aconitine and dendrotoxin subtracted to show only the % change in fluorescence induced by κA A671 itself. Values are mean+SEM from two individual trials. The response elicited by κA A671 alone (in the presence of aconitine) is also shown.

FIGS. 8A-8C show κA A671 is active in non-depolarized preparations. FIG. 8A shows the response (at 15 min) to increasing concentration of both peaks of κA A671 in cells not pretreated with a depolarizing agent. Data is from four individual trials for each Peak. FIG. 8B shows a comparison of the response of the cortical cell cultures of κA A671 peak 2 in depolarized vs. non-depolarized environment (data from 4-10 individual trials). FIG. 8C shows a comparison of the response induced by Peak 1 of κA A671 in depolarized vs. non-depolarized cells (data from 4-10 individual trials).

FIG. 9 shows 4-AP induces an increase in intracellular calcium in cells pretreated with Aconitine and in untreated cells. Values represent data (15 min exposure) from 2-3 individual trials. Data from depolarized environment are % change from pretreatment values and are corrected to the Aconitine-alone response.

### DETAILED DESCRIPTION OF THE PREFERRED EMBODIMENTS

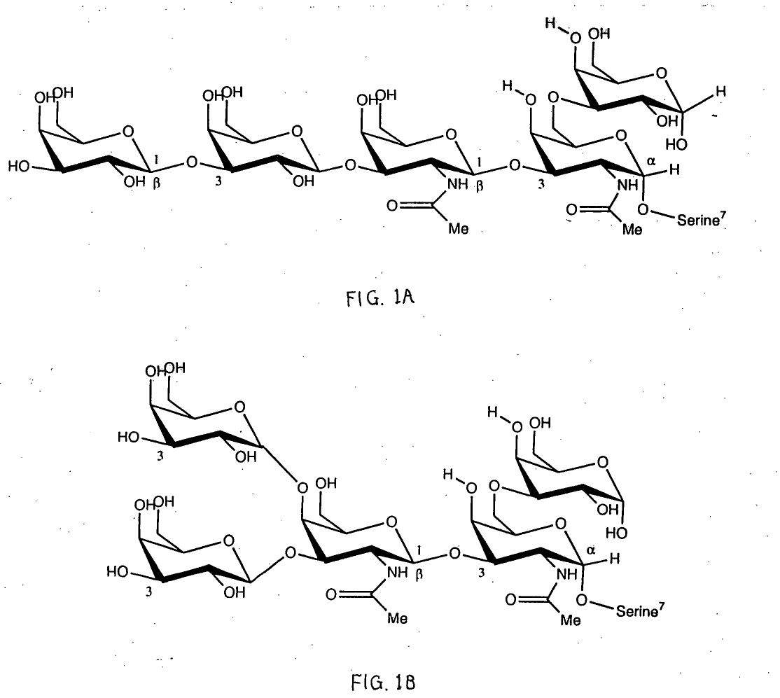

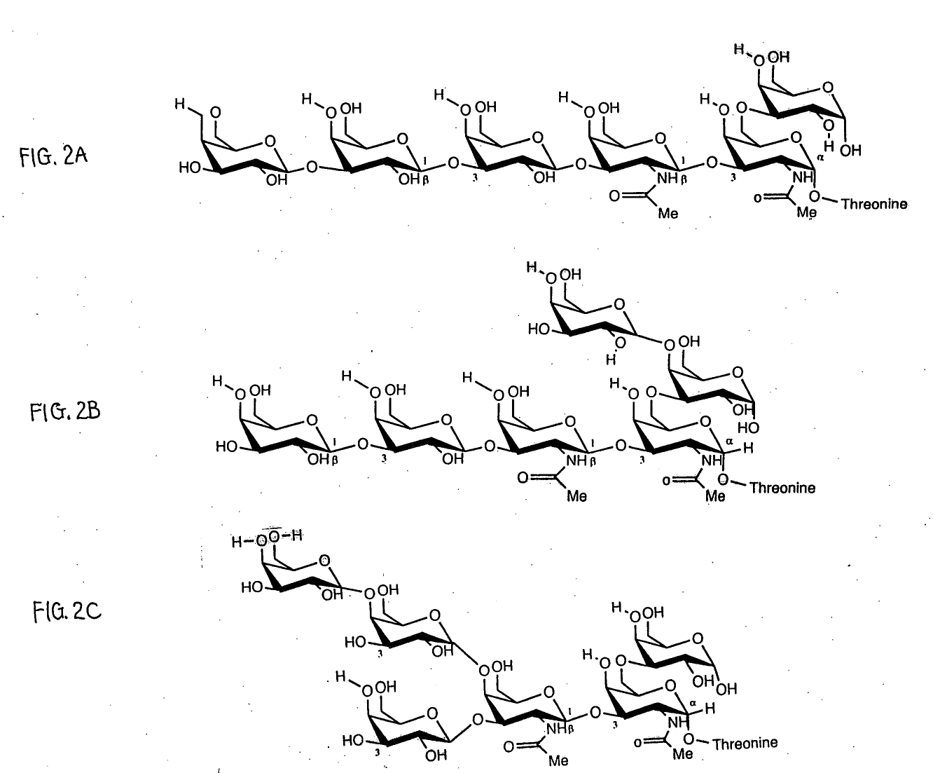

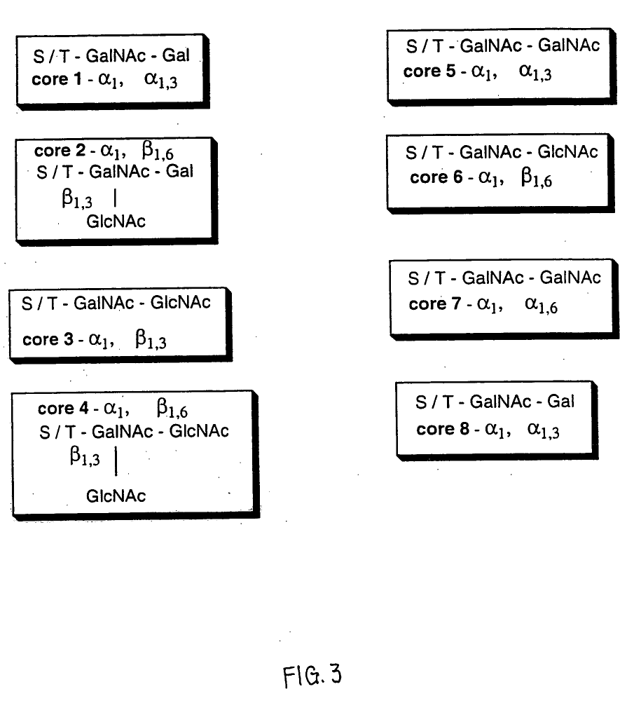

The present invention is directed to κA-conopeptides as described above. The κA-conopeptides may contain single or multiple O-glycan post-translational modifications at one or more, up to all, of the hydroxyl sites of the κA-conopeptides. The O-glycans are as described herein. Native O-glycans attached to κA SIVa and κA U006 are shown in FIGS. 1A-1B and FIGS. 2A-2C, respectively. The preferred core O-glycans which can be used to modify and of the κA-conopeptides disclosed herein are shown in FIG. 3. Further branching from these cores using the monosaccharides described herein may also be nade. Preferred glycosidic linkages are specified by cores 5 and 7 of FIG. 3 with further homolgation of the glycan at positions 3, 4 and 6 of the GalNAc template using the monosaccharides described herein Any free hydroxy funtion may be O-sulphated, O-posphorylated or O-aceylated. The disulfide bridges and activity of κA-conopeptides are described in U.S. Pat. No. 5,633,347.

The present invention is further directed to DNA sequences coding for several of these κA-conopeptides as described in further herein. The invention is further directed to propeptides for several of these κA-conopeptides as described in further detail herein.

Examples 1-5 describes the isolation and characterization of κA conotoxin SIVA. As described in these examples, κA SIVA elicits a spastic paralysis when injected into fish. When tested in a frog neuromuscular preparation, κA SIVA elicits a single muscle action potential from muscle. These results, as well as additional biological testing as described in these example, are consistent with blocking of potassium channels. Example 6 describes the isolation of additional κA conotoxins. Examples 7-12 describe the synthesis and characterization of the peptide κA A671. The biological testing for this peptide also demonstrates that the κA conopeptides block voltage-gated potassium channels. The biological testing described herein demonstrates that the κA conopeptides regulate flow of potassium ions and are useful for treating demylenating disorders, among other disorders as described herein.

Potassium channels comprise a large and diverse group of proteins that, through maintenance of the cellular membrane potential, are fundamental in normal biological function. The therapeutic applications for compounds that regulate the flow of potassium ions through K⁺ channels are far-reaching and include treatments of a wide range of disease and injury states. Disorders which can be treated using these conopeptides include multiple sclerosis, other demyelinating diseases (such as acute dissenmiated encephalomyelitis, optic neuromyelitis, adrenoleukodystrophy, acute transverse myelitis, progressive multifocal leukoencephalopathy), sub-acute sclerosing panencephalomyelitis (SSPE), metachromatic leukodystrophy, Pelizaeus-Merzbacher disease, spinal cord injury, botulinum toxin poisoning, Huntington's chorea, compression and entrapment neurophathies (such as carpal tunnel syndrome, ulnar nerve palsy), cardiovascular disorders (such as cardiac arrhythmias, congestive heart failure), reactive gliosis, hyperglycemia, immunosuppression, cocaine addiction, cancer, cognitive dysfunction, disorders resulting from defects in neurotransmitter release (such as Eaton-Lambert syndrome), and reversal of the actions of curare and other neuromuscular blocking drugs.

The κA conopeptides of the present invention are identified by isolation from Conusvenom. Alternatively, the κA conopeptides of the present invention are identified using recombinant DNA techniques by screening cDNA libraries of various Conus species using conventional techniques, such as the use of reverse-transcriptase polymerase chain reaction (RT-PCR) or the use of degenerate probes. Primers for RT-PCR are based on conserved sequences in the signal sequence and 3′ untranslated region of the A family conopeptide genes. Clones which hybridize to degenerate probes are analyzed to identify those which meet minimal size requirements, i.e., clones having approximately 300 nucleotides (for a propeptide), as determined using PCR primers which flank the cDNA cloning sites for the specific cDNA library being examined. These minimal-sized clones and the clones produced by RT-PCR are then sequenced. The sequences are then examined for the presence of a peptide having the characteristics noted above for κA-conopeptides. The biological activity of the peptides identified by this method is tested as described herein, in U.S. Pat. No. 5,635,347 or conventionally in the art.

These peptides are sufficiently small to be chemically synthesized. General chemical syntheses for preparing the foregoing conopeptides peptides are described hereinafter, along with specific chemical synthesis of conopeptides and indications of biological activities of these synthetic products. Various ones of these conopeptides can also be obtained by isolation and purification from specific Conus species using the techniques described in U.S. Pat. Nos. 4,447,356 (Olivera et al., 1984), 5,514,774 (Olivera et al., 1996) and 5,591,821 (Olivera et al., 1997), the disclosures of which are incorporated herein by reference.

Although the conopeptides of the present invention can be obtained by purification from cone snails, because the amounts of conopeptides obtainable from individual snails are very small, the desired substantially pure conopeptides are best practically obtained in commercially valuable amounts by chemical synthesis using solid-phase strategy. For example, the yield from a single cone snail may be about 10 micrograms or less of conopeptide. By “substantially pure” is meant that the peptide is present in the substantial absence of other biological molecules of the same type; it is preferably present in an amount of at least about 85% purity and preferably at least about 95% purity. Chemical synthesis of biologically active conopeptides depends of course upon correct determination of the amino acid sequence. Thus, the conopeptides of the present invention may be isolated, synthesized and/or substantially pure.

The conopeptides can also be produced by recombinant DNA techniques well known in the art. Such techniques are described by Sambrook et al. (1989). The peptides produced in this manner are isolated, reduced if necessary, and oxidized to form the correct disulfide bonds, if present in the final molecule.

One method of forming disulfide bonds in the conopeptides of the present invention is the air oxidation of the linear peptides for prolonged periods under cold room temperatures or at room temperature. This procedure results in the creation of a substantial amount of the bioactive, disulfide-linked peptides. The oxidized peptides are fractionated using reverse-phase high performance liquid chromatography (HPLC) or the like, to separate peptides having different linked configurations. Thereafter, either by comparing these fractions with the elution of the native material or by using a simple assay, the particular fraction having the correct linkage for maximum biological potency is easily determined. It is also found that the linear peptide, or the oxidized product having more than one fraction, can sometimes be used for in vivo administration because the cross-linking and/or rearrangement which occurs in vivo has been found to create the biologically potent conopeptide molecule. However, because of the dilution resulting from the presence of other fractions of less biopotency, a somewhat higher dosage may be required.

The peptides are synthesized by a suitable method, such as by exclusively solid-phase techniques, by partial solid-phase techniques, by fragment condensation or by classical solution couplings.

In conventional solution phase peptide synthesis, the peptide chain can be prepared by a series of coupling reactions in which constituent amino acids are added to the growing peptide chain in the desired sequence. Use of various coupling reagents, e.g., dicyclohexylcarbodiimide or diisopropylcarbonyldimidazole, various active esters, e.g., esters of N-hydroxyphthalimide or N-hydroxy-succinimide, and the various cleavage reagents, to carry out reaction in solution, with subsequent isolation and purification of intermediates, is well known classical peptide methodology. Classical solution synthesis is described in detail in the treatise, “Methoden der Organischen Chemie (Houben-Weyl): Synthese von Peptiden,” (1974). Techniques of exclusively solid-phase synthesis are set forth in the textbook, “Solid-Phase Peptide Synthesis,” (Stewart and Young, 1969), and are exemplified by the disclosure of U.S. Pat. No. 4,105,603 (Vale et al., 1978). The fragment condensation method of synthesis is exemplified in U.S. Pat. No. 3,972,859 (1976). Other available syntheses are exemplified by U.S. Pat. Nos. 3,842,067 (1974) and 3,862,925 (1975). The synthesis of peptides containing γ-carboxyglutamic acid residues is exemplified by Rivier et al. (1987), Nishiuchi et al. (1993) and Zhou et al. (1996). Synthesis of conopeptides have been described in U.S. Pat. Nos. 4,447,356 (Oliveraet al., 1984), 5,514,774 (Oliveraet al., 1996) and 5,591,821 (Olivera et al., 1997).

Common to such chemical syntheses is the protection of the labile side chain groups of the various amino acid moieties with suitable protecting groups which will prevent a chemical reaction from occurring at that site until the group is ultimately removed. Usually also common is the protection of an α-amino group on an amino acid or a fragment while that entity reacts at the carboxyl group, followed by the selective removal of the α-amino protecting group to allow subsequent reaction to take place at that location. Accordingly, it is common that, as a step in such a synthesis, an intermediate compound is produced which includes each of the amino acid residues located in its desired sequence in the peptide chain with appropriate side-chain protecting groups linked to various ones of the residues having labile side chains.

As far as the selection of a side chain amino protecting group is concerned, generally one is chosen which is not removed during deprotection of the α-amino groups during the synthesis. However, for some amino acids, e.g., His, protection is not generally necessary. In selecting a particular side chain protecting group to be used in the synthesis of the peptides, the following general rules are followed: (a) the protecting group preferably retains its protecting properties and is not split off under coupling conditions, (b) the protecting group should be stable under the reaction conditions selected for removing the α-amino protecting group at each step of the synthesis, and (c) the side chain protecting group must be removable, upon the completion of the synthesis containing the desired amino acid sequence, under reaction conditions that will not undesirably alter the peptide chain.

It should be possible to prepare many, or even all, of these peptides using recombinant DNA technology. However, when peptides are not so prepared, they are preferably prepared using the Merrifield solid-phase synthesis, although other equivalent chemical syntheses known in the art can also be used as previously mentioned. Solid-phase synthesis is commenced from the C-terminus of the peptide by coupling a protected α-amino acid to a suitable resin. Such a starting material can be prepared by attaching an α-amino-protected amino acid by an ester linkage to a chloromethylated resin or a hydroxymethyl resin, or by an amide bond to a benzhydrylamine (BHA) resin or para-methylbenzhydrylamine (MBHA) resin. Preparation of the hydroxymethyl resin is described by Bodansky et al. (1966). Chloromethylated resins are commercially available from Bio Rad Laboratories (Richmond, Calif.) and from Lab. Systems, Inc. The preparation of such a resin is described by Stewart and Young (1969). BHA and MBHA resin supports are commercially available, and are generally used when the desired polypeptide being synthesized has an unsubstituted amide at the C-terminus. Thus, solid resin supports may be any of those known in the art, such as one having the formulae —O—CH₂-resin support, —NH BHA resin support, or —NH-MBHA resin support. When the unsubstituted amide is desired, use of a BHA or MBHA resin is preferred, because cleavage directly gives the amide. In case the N-methyl amide is desired, it can be generated from an N-methyl BHA resin. Should other substituted amides be desired, the teaching of U.S. Pat. No. 4,569,967 (Kornreich et al., 1986) can be used, or should still other groups than the free acid be desired at the C-terminus, it may be preferable to synthesize the peptide using classical methods as set forth in the Houben-Weyl text (1974).

The C-terminal amino acid, protected by Boc or Fmoc and by a side-chain protecting group, if appropriate, can be first coupled to a chloromethylated resin according to the procedure set forth in Horiki et al. (1978), using KF in DMF at about 60° C. for 24 hours with stirring, when a peptide having free acid at the C-terminus is to be synthesized. Following the coupling of the BOC-protected amino acid to the resin support, the a-amino protecting group is removed, as by using trifluoroacetic acid (TFA) in methylene chloride or TFA alone. The deprotection is carried out at a temperature between about 0° C. and room temperature. Other standard cleaving reagents, such as HCl in dioxane, and conditions for removal of specific a-amino protecting groups may be used as described in Schroder and Lubke (1965).

After removal of the a-amino-protecting group, the remaining α-amino- and side chain-protected amino acids are coupled step-wise in the desired order to obtain the intermediate compound defined hereinbefore, or as an alternative to adding each amino acid separately in the synthesis, some of them may be coupled to one another prior to addition to the solid phase reactor. Selection of an appropriate coupling reagent is within the skill of the art. Particularly suitable as a coupling reagent is N,N′-dicyclohexylcarbodiimide (DCC, DIC, HBTU, HATU, TBTU in the presence of HoBt or HoAt).

The activating reagents used in the solid phase synthesis of the peptides are well known in the peptide art. Examples of suitable activating reagents are carbodiimides, such as N,N′-diisopropylcarbodiimide and N-ethyl-N′-(3-dimethylaminopropyl)carbodiimide. Other activating reagents and their use in peptide coupling are described by Schroder and Lubke (1965) and Kapoor (1970).

Each protected amino acid or amino acid sequence is introduced into the solid-phase reactor in about a twofold or more excess, and the coupling may be carried out in a medium of dimethylformamide (DMF):CH₂Cl₂ (1:1) or in DMF or CH₂Cl₂ alone. In cases where intermediate coupling occurs, the coupling procedure is repeated before removal of the α-amino protecting group prior to the coupling of the next amino acid. The success of the coupling reaction at each stage of the synthesis, if performed manually, is preferably monitored by the ninhydrin reaction, as described by Kaiser et al. (1970). Coupling reactions can be performed automatically, as on a Beckman 990 automatic synthesizer, using a program such as that reported in Rivier et al. (1978).

After the desired amino acid sequence has been completed, the intermediate peptide can be removed from the resin support by treatment with a reagent, such as liquid hydrogen fluoride or TFA (if using Fmoc chemistry), which not only cleaves the peptide from the resin but also cleaves all remaining side chain protecting groups and also the α-amino protecting group at the N-terminus if it was not previously removed to obtain the peptide in the form of the free acid. If Met is present in the sequence, the Boc protecting group is preferably first removed using trifluoroacetic acid (TFA)/ethanedithiol prior to cleaving the peptide from the resin with HF to eliminate potential S-alkylation. When using hydrogen fluoride or TFA for cleaving, one or more scavengers such as anisole, cresol, dimethyl sulfide and methylethyl sulfide are included in the reaction vessel.

Cyclization of the linear peptide is preferably affected, as opposed to cyclizing the peptide while a part of the peptido-resin, to create bonds between Cys residues. To effect such a disulfide cyclizing linkage, fully protected peptide can be cleaved from a hydroxymethylated resin or a chloromethylated resin support by ammonolysis, as is well known in the art, to yield the fully protected amide intermediate, which is thereafter suitably cyclized and deprotected. Alternatively, deprotection, as well as cleavage of the peptide from the above resins or a benzhydrylamine (BHA) resin or a methylbenzhydrylamine (MBHA), can take place at 0° C. with hydrofluoric acid (HF) or TFA, followed by oxidation as described above. A suitable method for cyclization is the method described by Cartier et al. (1996).

Muteins, analogs or active fragments, of the foregoing τ-conotoxin peptides are also contemplated here. See, e.g., Hammerland et al (1992). Derivative muteins, analogs or active fragments of the conotoxin peptides may be synthesized according to known techniques, including conservative amino acid substitutions, such as outlined in U.S. Pat. No. 5,545,723 (see particularly col. 2, line 50 to col. 3, line 8); U.S. Pat. No. 5,534,615 (see particularly col. 19, line 45 to col. 22, line 33); and U.S. Pat. No. 5,364,769 (see particularly col. 4, line 55 to col. 7, line 26), each incorporated herein by reference.

Pharmaceutical compositions containing a compound of the present invention as the active ingredient can be prepared according to conventional pharmaceutical compounding techniques. See, for example, Reminton 's Pharmaceutical Sciences, 18th Ed. (1990, Mack Publishing Co., Easton, Pa.). Typically, an antagonistic amount of the active ingredient will be admixed with a pharmaceutically acceptable carrier. The carrier may take a wide variety of forms depending on the form of preparation desired for administration, e.g., intravenous, oral or parenteral. For examples of delivery methods, see U.S. Pat. No. 5,844,077, incorporated herein by reference.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media maybe employed, such as, for example, water, glycols, oils, alcohols, flavoring agents, preservatives, coloring agents, suspending agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable for passage through the gastrointestinal tract, while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered, and the rate and time-course of administration, will depend on the nature and severity of the condition being treated. Prescription of treatment, e.g. decisions on dosage, timing, etc., is within the responsibility of general practitioners or specialists, and typically takes into account the disorder to be treated, the condition of the individual patient, the site of delivery, the method of administration and other factors known to practitioners. Examples of techniques and protocols can be found in Remington 's Pharmaceutical Sciences. Typically, the active agents of the present invention exhibit their effect at a dosage range of from about 0.001 mg/kg to about 250 mg/kg, preferably from about 0.01 mg/kg to about 100 mg/kg, of the active ingredient and more preferably, from about 0.05 mg/kg to about 75 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved.

Alternatively, targeting therapies may be used to deliver the active agent more specifically to certain types of cells, by the use of targeting systems such as antibodies or cell-specific ligands. Targeting maybe desirable for a variety of reasons, e.g. if the agent is unacceptably toxic, if it would otherwise require too high a dosage, or if it would not otherwise be able to enter target cells.

The active agents, which are peptides, can also be administered in a cell-based delivery system in which a DNA sequence encoding an active agent is introduced into cells designed for implantation in the body of the patient, especially in the spinal cord region. Suitable delivery systems are described in U.S. Pat. No. 5,550,050 and in published PCT Applications No. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of developed sequences and the known genetic code.

### EXAMPLES

the present invention is described by reference to the following Examples, which are offered by way of illustration and are not intended to limit the invention in any manner. Standard techniques well known in the art or the techniques specifically described below were utilized.

### Example 1

### Materials and Methods for Kappa-A SIVA Isolation and Analysis

Venom collection; Bioassay. Specimens of Conus striatus were collected in the Philippines. The molluscs were buried in ice for 30 min, the venom apparatus dissected and venom scraped from the duct. Animals for bioassay included mice (Japanese sDDy or Swiss Webster) and fish.

Purification.

Several different batches of the peptide were purified from crude Conus striatus venom using two different methods. Most studies were originally done on material purified by Purification II; however, Purification I has been used as the routine method for obtaining more recent batches of the peptide.

Purification I. Crude venom from dissected ducts of C. striatus was pooled and stored at −70° C. Venom (50 mg) was placed in an Eppendorf tube and 0.5% trifluoroacetic acid in distilled, deionized water was added (1.5 mL, 0 ° C.). The tube was placed in ice for 20 min. It was vortexed for 5 min, then centrifuged at 20,000 rpm using an SM-24 rotor in a Sorvall RC2-B centrifuge for 30 min at 4° C. The supernatant was collected and 0.5% trifluoroacetic acid (1.5 mL) was added to the remaining pellet; the procedure was repeated again for a second extraction. The two supernatants were then combined.

Crude venom extract (0.5 mL) was run on an analytical Vydac C₁₈ column with a guard cartridge; the active fractions from six runs were pooled. The peptide was further purified by running it again on the analytical Vydac C₁₈ column without the guard column. For all HPLC chromatography, a gradient from 0.1% trifluoroacetic acid to 0.09% trifluoroacetic acid and 60% acetonitrile was used with a linear increase for acetonitrile of 0.6%/min. Trifluoroacetic acid (sequencing grade) and acetonitrile (HPLC grade) were obtained from Fisher.

Purification II. Lyophilized venom (˜0.5 g) was suspended in 1.1% acetic acid (2.0 mL) and stirred, then placed on ice for 30 min and centrifuged at 10,000 rpm (Sorvall SS-34) for 10 min. The supernatant was collected; the pellet was redissolved in the same solvent, sonically disrupted five times for 10 s with 10 s intervals (60-70 W setting, Sonifier Cell Disruptor model WI 85 equipped with a microtip). Centrifugation followed, and the above procedure was repeated on the pellet. All three supernatants were combined and lyophilized to provide the crude venom extract which was lyophilized and dissolved in 10 mL of 1.1% acetic acid, applied to a Sephadex G-25 column (110×2.5 cm) and eluted with 1.1% acetic acid inside the LKB Min Cold Lab set at 5 ° C. Blue dextran and bacitracin (Mᵣ=2×10⁶ and 1,400, respectively) were used as standards. Fractions (10 mL) were collected at a flow rate of 0.27 mL/min.

Fractions from Sephadex G-25 chromatography exhibitingbiological activity were pooled, lyophilized and refractionated by reversed-phase HPLC using an Ultropac TSK ODS-120T semi-preparative column (7.8×300 mm, 10 μm particle size, fully capped). Peptides were eluted with a linear gradient of acetonitrile in 0.1% trifluoroacetic acid (solvent A, 0.1% trifluoroacetic acid and solvent B, 0.1% trifluoroacetic acid in 60% acetonitrile) at a flow rate of 2 mL/min. Bioactive fractions were rechromatographed 2-3 times, as needed, on an analytical reversed-phase C₁₈ column to remove contaminants. Elution was done with a gradient of acetonitrile in 0.05% heptafluorobutyric acid (solvent A, 0.05% heptafluorobutyric acid, and solvent B, 0.05% heptafluorobutyric acid in 60% acetonitrile) at a flow rate of 1 mL/min. Absorbance of the effluent was monitored at 214 nm and fractions were collected manually.

Bioassay. Lyophilized venom extracts and column fractions were resuspended in NSS. For mice (10 g), the fractions were injected intraperitoneally (i.p.) and intracranially (i.c.) and the animals were monitored for peculiar movements and neurological manifestations. Fish (1-2.5 g) were injected i.p. with toxin solution (5 μL) using a 10 μl Hamilton syringe in the ventral area between the anal fin and the pelvic fins.

Other Methods. The protein content of the venom samples and fractions were determined according to the method of Lowry et al. (1951), with bovine serum albumin serving as a standard.

Proteolysis was carried out using purified toxin (2.55 μg of protein) dissolved in 12 μL of 0.05M N-ethyl morpholine acetate, pH 8.9, 0.5 nM CaCl₂ containing 0.2 mg/mL trypsin or α-chymotrypsin. Control toxin solutions containing no proteolytic enzymes were also prepared. Samples were incubated at 37 ° C. for 4 h then diluted 2-fold with distilled water. Aliquots were assayed for toxicity in fish. At least three fish were injected for each sample and kept under observation for 8 h.

The toxin was reduced by using purified toxin (2.39 μg of protein) dissolved in 20 μL of β-mercaptoethanol (30 μL of β-mercaptoethanol in 200 μL of distilled water). Control toxin solutions containing no reducing agent were also prepared. Samples were incubated at room temperature under nitrogen for 4 h. Aliquots were assayed for toxicity on fish. At least three fish were tested at each dose.

Amino Acid Analysis and Sequencing. Amino acid analyses and sequencing were carried out at both the University of Utah Biology Department and the Salk Institute to yield a single consistent sequence.

Amino acid analysis was carried out using the Waters PICO.TAG amino acid analysis system. The peptide samples were first hydrolyzed with 6N HCl and then derivatized with phenylisothiocyanate to produce phenylthiocarbamyl amino acids which were separated by HPLC. Molar ratios were compared based on amino acid analysis assuming that the amino acids with the lowest percentage are represented once in the polypeptide. Sequence analysis of peptides was carried out by sequential Edman degradation in a Beckman 890D spinning cup sequencer, using the 0.1M Quadrol Program. Peptide fragments were analyzed by a manual method. Phenylthiohydantoin-amino acids were analyzed by HPLC.

Mass Spectrometry. Liquid secondary ionization (Barber et al., 1982) mass spectra (LSI-MS) were measured using a JEOLHX110 (JEOL, Tokyo, Japan) double focusing mass spectrometer operated at 10 kV accelerating voltage. The sample (in 0.1% aqueous trifluoroacetic acid and 25% acetonitrile) was mixed in a glycerol, 3-nitrobenzyl alcohol matrix (1: 1). The LSI-MS spectra were measured with electric field scans at a nominal resolution of 1000. Electrospray mass spectra (ESI-MS) were measured using either an Esquire-LC (Bruker Daltonics, Billerica, MA) or an LCQ (Finnigan MAT, San Jose, Calif.) ion trap mass spectrometer. The peptide (0.1% aqueous trifluoroacetic acid diluted with 1% acetic acid in methanol) was analyzed by direct infusion. The mass range of the MS/MS spectrum was limited to 380-1850 Da.

Electrophysiology. Synaptically evoked responses from the cutaneus pectoris muscle of frog were performed as previously described (Shon, K-J. et al., 1998; Yoshikami, D. et al., 1989). Briefly, a pair of extracellular electrodes were used to stimulate the nerve. A wire electrode placed near the end plate of the muscle and reference electrode placed at the myotendenous end were connected to a differential amplifier to record extracellular responses from the muscle.

Intracellular recording of antidromic action potentials from neurons in intact sympathetic ganglia of the frog was performed as described by others (Dodd, J. et al., 1983). Briefly, an intracellular glass microelectrode (˜20 MΩ) measured the membrane potential from the soma of a neuron while the postganglionic nerve was stimulated with a suction electrode. On the other hand, to measure voltage-gated currents dissociated ganglionic neurons were prepared and whole-cell clamped with patch electrodes.

Whole-cell voltage clamp of Xenopus injected with cRNA was performed as previously described (see Shon, K-J. et al., 1998).

### Example 2

### Purification of the κA SIVA

A fraction of Conus striatus venom which induced a spastic paralysis in fish was further resolved by reversed-phase HPLC. This activity was purified to homogeneity as follows: The extract was chromatographed in 1.1% acetic acid. Peak B was chromatographed on an Ultropac TSK ODS-120T C₁₈ semipreparative column in trifluoroacetic acid with an acetonitrile gradient. BI was chromatographed on a second Ultropac column in hexafluorobutyric acid with an acetonitrile gradient. Sephadex and HPLC chromatography was performed as described in Purification II, previously. The purified activity was provisionally called the “spastic peptide” because of the symptomatology observed in fish.

Results of bioactivity assays of the spastic peptide are shown in Table 1. When injected i.p. in fish, the peptide induced a period ofrapid swimming followed by a spastic paralysis with stiff fibrillating fins. At sufficiently high doses, the peptide was lethal to both fish (i.p.>50 pmole/g) and mice (i.c.>400 pmole/g).

TABLE 1
Bioassay of Spastic Peptide

| Dose | |
|---|---|
| (pmol/g) | Observations |
| A. Goldfish (i.p.) | |
| 5 | No visible effect. |
| 10-15 | Hyperactivity ˜10 min; darting, tilted swimming ˜20 |
| | min; partial paralysis, fins fibrillating ˜180 min. |
| 50-55 | Darting, mouth open wider ˜4 min; paralysis; fins |
| | fibrillating ˜10 min; death ˜100 min. |
| >500 | Fins spread out, mouth open wider ˜2 min; paralysis, |
| | fins fibrillating ˜5 min; death 15 min. |
| B. Mice (i.c.v.) | |
| 50-100 | No visible effect. |
| >400 | Weak ˜20 min; can't stand upright ˜25 min; labored |
| | breathing ˜30 min; death ˜40 min. |

### Example 3

### Biochemical Characterization of κA SIVA

The purified spastic peptide was analyzed by liquid secondary ionization-mass spectrometry (LSI-MS); two intact species at m/z 4084.2 and 4100.5 were observed. Observation of species separated by 16 Da is often indicative of the sample containing a mixture of peptides with methionine and methionine sulfoxide generated upon standing. The sample was subjected to Edman degradation, but no sequence could be determined, suggesting that the peptide was blocked at the N-terminus. When treated with pyroglutamate aminopeptidase to unblock the peptide, sequence analysis gave the partial sequence KSLVPXVITTXXGYDOGTMXOOXRXTN (SEQ ID NO:45; X is unknown), where the level of signal-to-noise after cycle 27 did not allow unambiguous determination ofthe PTH amino acid in the remaining cycles. After reduction and pyridylethylation, five of the six blank cycles were resolved to give the partial sequence KSLVPXVITTCCGYDOGTMCOOCRC (SEQ ID NO:46; X is unknown). Microheterogeneity was observed in position 2 of the des-pyroglutamyl peptide, depending on the batch of venom used, with either a Ser (as above) or Glu residue present at this position.

Treatment of the reduced and alkylated peptide with protease Asp-N yielded three major fragments, two hydrophilic and one hydrophobic. The C-terminus of the peptide was determined by chemical sequencing and LSI-MS analysis ofthe two hydrophilic fragments. For both fragments the sequence DOGTMCOOCRCTNSC (residues 15-25 of SEQ ID NO:46) was obtained. The observed masses (m/z 2053.8 and 2069.2) indicated that the peptide was C-terminally amidated. On the basis of presence of the methionine residue, the two species in this fragment were assigned as methionine- and methionine-sulfoxide- containing analogs (Cf. calculated [M+H]⁺ average masses of 2053.5 and 2069.4 Da). The hydrophobic fragment was identified as the N-terminal fragment based upon the shift in retention time observed after pyroglutamate aminopeptidase treatment. Chemical sequencing of the N-terminal fragment also gave a blank cycle at the seventh position from the N-terminus suggesting the presence of a nonstandard amino acid. While three serine residues were detected in the peptide by amino acid analysis, only two serine residues were found using Edman degradation, suggesting the presence of an additional serine residue (modified) at position 7 and the following sequence:

*
XKSLVPSVITTCCGYDOGTMCOOCRCTNSC-NH₂ (SEQ ID NO:47)

(where [symbol] is a modified Ser, X is pyroglutamate and O is 4-hydroxyproline)

This assignment was verified by sequencing a cDNA clone encoding the peptide (results not shown); the nucleic acid sequence specified a serine codon at position 7.

### Example 4

### MS Evidence for Glycosylation of κA SIVA

The significant difference (δ=893.5 Da) between the mass of the intact peptide (m/z 4084.2) determined by LSI-MS and that predicted by the proposed sequence (3190.7 Da) suggested that the serine residue was post-translationally modified. Inspection of the ESI-MS/MS spectrum of the [M+3H]³⁺ parent ion revealed several features which indicate that the spastic peptide is glycosylated. The m/z 407.3, 568.9, 730.9 and 893.1 species are singly-charged fragment ions with masses which correspond with HexNAc₂ (406.8 Da), HexHexNAc₂ (568.8 Da), Hex₂HexNAc₂ (730.8 Da), and Hex₃HexNAc₂ (892.8 Da). The triply-charged fragment ions observed at m/z 1307.7, 1253.1 and 1199.5 are consistent with the loss of hexose residues from the intact ion while the doubly-charged ions observed at m/z 1778.1, 1696.9 and 1595.1 correspond with loss of Hex₂HexNAc, Hex₃HexNAc and Hex₃HexNAc₂. Fragment ions involvingpeptide chain cleavage were also observed in the mass spec at m/z 539.2, 1026.7, 1127.7, 1529.3, 1611.0, 1676.1 and 1772.0. An extended mass range MS/MS scan (m/z>1850) verified the general trends observed in the mass spec and revealed that a m/z 1859 doubly charged fragment ion is due to loss of hexose from the [M+3H]³⁺ ion. These results are consistent with an O-glycosylated serine residue present in position 7. The composition and sequence of the glycan are presently being determined, but the mass increment and fragmentation are consistent with Hex₃HexNAc₂ (892.817 Da).

### Example 5

### Electrophysiological Studies of κA SIVA

The spastic peptide was tested on the frog neuromuscular preparation. A single stimulus to the nerve invariably elicited only a single muscle action potential from the muscle. However, when the spastic peptide (100 nM) was present, a train of action potentials was elicited instead. Exposure to spastic peptide also produced spontaneous activity. Intracellularly recorded action potentials were also examined in intact frog sympathetic ganglia. Action potentials under control conditions were obtained by antidromic stimulation of the post-ganglionic nerve. Exposure to 100 nM peptide produced spontaneous action potentials; compared to controls, these had a wider overshooting, depolarizing phase and no undershoot. All these characteristics are consistent with blocking ofpotassium channels. Furthermore, in preliminary voltage-clamp experiments with TTX-treated dissociated neurons from the ganglion, outward currents elicited by step depolarizations (to −30 mV or more from a −70 mV holding potential) were attenuated by 3 μM toxin.

The spastic peptide is an antagonist of cloned Shaker K⁺ channels. The block of K⁺ currents produced by the peptide was only slowly reversible. Together, the data strongly indicate that the spastic peptide is a potassium channel blocker. We have designated the spastic peptide as the first member of a new family of Conus peptides; the peptide described here is designated κA-conotoxin SIVA, consistent with the nomenclature previously used in the Conus peptide system.

The data presented in Examples 1-5 detail the purification and characterization of a novel Conus peptide, κA conotoxin SIVA which elicits a spasticparalysis when injected into fish. Among the features of Conus venoms characterized so far, a distinguishing electrophysiological hallmark of the peptide is its ability to elicit repetitive action potentials in the frog nerve-muscle preparation. The neuroexcitatory activity of the peptide is due to blockage of voltage-gated potassium channels. More specifically, the peptide appears to contribute to the excitotoxic shock symptomatology observed when Conus striatus stings a fish; it is the single most potent (pmole/g) excitotoxic peptide thus far observed when administered i.p. in fish.

At the biochemical level, there are striking differences between this peptide, and other previously characterized Conus peptides. Two unique features are the relatively long N-terminal region (11 AA) preceding the first disulfide linkage and the presence of an O-glycosylated serine residue at position 7. This post-translational modification has not previously been observed in Conus peptides. The blocked amino terminus, the presence of three disulfide bridges, a methionine residue and the N-terminal extension present in SIVA are all features which are observed in Charbydotoxin type (α-KT×1) scorpion toxins, where five amino acids separate the pyroglutamic acid residue from the N-terminal cysteine residue. However, the absence of charged residues in the SIVA cysteine rich domain structure is in contrast with both κ-conotoxin PVIIA (Shon et al., 1998) and the scorpion K⁺ channel toxins (Miller, 1995).

Like most biologically active peptides in Conus venoms, κA conotoxin SIVA has multiple disulfide bonds. The arrangement and spacing of all but one of the six Cys residues is similar to that of the αA-conotoxins EIVA and PIVA (Hopkins et al, 1995; Jacobsen et al., 1997).

A conserved motif is observed in all three peptides; in addition, two hydroxyproline and one glycine residue are conserved in all three peptides. Like those ofthe αA-conotoxins, all proline residues in between disulfide linkages are hydroxylated; however, in κA conotoxin the proline residue in the N-terminal regional tail region remains unmodified. In addition, although the αA-conotoxins are competitive nicotinic receptor antagonists, we note that κA conotoxin is clearly a K⁺ channel antagonist.

As described further below in Example 6, similar peptides are present in other Indo-Pacific fish-hunting Conus species. Homologs of κA conotoxin SIVA were found in Conus magus, Conus stercusmuscarum, Conus circumcisus and Conus striolatus, suggesting a Conus peptide family widely distributed in hook-and-line piscivorous Conus from the Indo-Pacific. The κA conotoxin is a further example of a peptide which illustrates the distinction between the molecular pharmacology of prey capture in Indo-Pacific and non-Indo-Pacific fish-hunting Conus species. We have attempted to identify a spastic peptide homolog in Conus purpurascens venom without success. It appears to be absent, both from an analysis of the venom and of a cDNA library of this species. Thus, κA conotoxin SIVA is the first biochemically-characterized member of a family of Conus peptides which is widely distributed in hook-and-line fish-hunting Indo-Pacific Conus.

Like κA SIVA, vespulakinin I and II which are glycopeptides isolated from yellow jacket wasps (Vespula maculifrons) (Yoshida et al., 1976) are polypeptide constituents of venoms. The sites ofglycosylation for vespulakinin and αA-conotoxin are consistent with the very general motifs of O-linked glycosylation found previously for glycophorin (Piscano et al., 1993). The nine C-terminal amino acids of the vespulakinins code for the neuropeptide bradykinin. Comparison of synthetic glycosylated and nonglycosylated vespulakinin analogues indicate that the glycosylated analogue is more active in stimulating guinea pig rectum contraction than the nonglycosylated analogue (Gobbo et al., 1992). Similarly, preliminary results with synthetic nonglycosylated κA-conotoxin analogues indicate that these are far less potent when injected into animals than are the glycosylated κA-conotoxins.

We suggest that αA-conotoxin SIVA plays a role analogous to that of αA-conotoxin PVIIA for C. purpurascens, i.e., it is one of the major venom components involved in the physiological strategy of the cone snail for eliciting excitotoxic shock in its fish prey that results in immediate immobilization. Accordingly, in vivo κA-conotoxin must be able to incapacitate the appropriate target K⁺ channels extremely rapidly. Thus, a plausible role for the glycosylation is either increasing the on-time and/or affinity of the peptide for its target K⁺ channel or increasing the speed of access of the peptide to its target K⁺ channels.

### Example 6

### Identification of Additional κA Conopeptides

The κA conopeptides MVIIA, SM1 and SM2, as well as their propeptides, were isolated as described in U.S. Pat. No. 5,633,347, incorporated herein by reference.

Additional κA conopeptides were identified by cloning by reverse transcription-polymerase chain reaction (RT-PCR) from cone snail venom duct mRNA. The PCR primers were based on conserved sequences in the signal sequence and 3′ untranslated regions of the A family conopeptide genes. The sequences of the primers used for cloning were:

forward primer A Con7:

(SEQ ID NO:48)

CAGGATCCATGTTCACCGTGTTTCTGTTGG
and
reverse primer KACon1:

(SEQ ID NO:49)

ATCTCGAGCATCAGTCGTTTCTGCG.

RT-PCR of venom duct mRNA produces a product of about 250 nucleotides in Conus species that express κA genes. The PCR product is then cloned into a plasmid vector and individual clones are sequenced to determine the sequence of various κA genes. In this manner, κA peptides were cloned from Conus aurisiacus and Conus consors. The DNA sequence and corresponding protein sequences are set forth in Tables 2-5.

TABLE 2
DNA (SEQ ID NO:50) and Protein (SEQ ID NO:51) Sequence of κA A671

atg ttc acc gtg ttt ctg ttg gtt gtc ttg gca acc act gtc gtt tcc
Met Phe Thr Val Phe Leu Leu Val Val Leu Ala Thr Thr Val Val Ser
atc cct tca gat cgt gca tct gat ggc agg aat gcc gca gtc aac gag
Ile Pro Ser Asp Arg Ala Ser Asp Gly Arg Asn Ala Ala Val Asn Glu
aga gcg cct tgg ctg gtc cct tcg aca atc acg act tgc tgt gga tat
Arg Ala Pro Trp Leu Val Pro Ser Thr Ile Thr Thr Cys Cys Gly Tyr
aat ccg ggg aca atg tgc cct cct tgc agg tgc gat aat acc tgt
Asn Pro Gly Thr Met Cys Pro Pro Cys Arg Cys Asp Asn Thr Cys
taaccaaaaa aaaccaaaac caggccgcag aaacgactga tgctccagga ccctctgaac
cacgacatgc cgccctctgc ctgacctgct tcactttccg tctctttgtg ccactagaac
tgtacaactc gatccactag actcccacgt tacctccgta ttctgaaact acttggattt
gattgtcctt aatatctgct catacttgct gttattacat cgtccaaaaa aaaaaaaaaa
aaaaa

TABLE 3

DNA (SEQ ID NO:52) and Protein (SEQ ID NO:53)
Sequence of κA H350

ggatcc atg ttc acc gtg ttt ctg ttg gtt gtc ttg gca acc act gtc
Met Phe Thr Val Phe Leu Leu Val Val Leu Ala Thr Thr Val
gtt tcc atc cct tca gat cgt gca tct gat ggc agg aat gcc gca gtc
Val Ser Ile Pro Ser Asp Arg Ala Ser Asp Gly Arg Asn Ala Ala Val
aac gag aga caa tct tgg ctg gtc cct tcg aca atc acg act tgc tgt
Asn Glu Arg Gln Ser Trp Leu Val Pro Ser Thr Ile Thr Thr Cys Cys
gga tat gat ccg ggg aca atg tgc cct cct tgc agg tgc aat aat acc
Gly Tyr Asp Pro Gly Thr Met Cys Pro Pro Cys Arg Cys Asn Asn Thr
tgt aaa cca aaa aaa cca aaa cca gga aaa ggc cgc aga aac gac
Cys Lys Pro Lys Lys Pro Lys Pro Gly Lys Gly Arg Arg Asn Asp
tgatgctcca ggaccctctg aaccacgacc tcgag

TABLE 4

DNA (SEQ ID NO:54) and Protein (SEQ ID NO:55)
Sequence of κA J454

ggatcc atg ttc acc gtg ttt ctg ttg gtt gtc ttg gca acc act gtc
Met Phe Thr Val Phe Leu Leu Val Val Leu Ala Thr Thr Val
gtt tcc atc cct tca gat cgt gca tct gaa ggc agg aat gcc gta gtc
Val Ser Ile Pro Ser Asp Arg Ala Ser Glu Gly Arg Asn Ala Val Val
cac gag aga gcg cct gag ctg gtc gtt acg gcc acc acg act tgc tgt
His Glu Arg Ala Pro Glu Leu Val Val Thr Ala Thr Thr Thr Cys Cys
ggt tat gat ccg atg aca ata tgc cct cct tgc atg tgc act cat tcc
Gly Tyr Asp Pro Met Thr Ile Cys Pro Pro Cys Met Cys Thr His Ser
tgt cca cca aaa aga aaa cca ggc cgc aga aac gac tgatgctcga g
Cys Pro Pro Lys Arg Lys Pro Gly Arg Arg Asn Asp

TABLE 5

DNA (SEQ ID NO:56) and Protein (SEQ ID NO:57)
Sequence of κA G851

ggatcc atg ttc acc gtg ttt ctg ttg gtt gtc ttg gca acc act gtc
Met Phe Thr Val Phe Leu Leu Val Val Leu Ala Thr Thr Val
gtt tcc atc cct tca gat cgt gca tct gat ggc agg aat gcc gta gtc
Val Ser Ile Pro Ser Asp Arg Ala Ser Asp Gly Arg Asn Ala Val Val
cac gag aga gcg cct gag ctg gtc gtt acg gcc acc acg act tgc tgt
His Glu Arg Ala Pro Glu Leu Val Val Thr Ala Thr Thr Thr Cys Cys
ggt tat gat ccg atg aca tgg tgc cct tct tgc atg tgc act tat tcc
Gly Tyr Asp Pro Met Thr Trp Cys Pro Ser Cys Met Cys Thr Tyr Ser
tgt ccc cac caa agg aaa aaa cca ggc cgc aga aac gac tgatgctcca
Cys Pro His Gln Arg Lys Lys Pro Gly Arg Arg Asn Asp
ggaccctctg aaccacgacc tcgag

In a similar procedure, κA conopeptides were cloned from Conus achatinus, Conus catus, Conus circumcisus, Conus consors, Conus magus, Conus monachus, Conus stercusmuscarum, Conus striatus, Conus striolatus and Conus sulcatus. The DNA sequence and corresponding protein sequences are set forth in Tables 6-25.

TABLE 6

DNA (SEQ ID NO:58) and Protein (SEQ ID NO:59)
Sequence of κA A10.1

atg ttc acc gtg ttt ctg ttg gtt gtc ttg gca acc act ctc gtt tcc
Met Phe Thr Val Phe Leu Leu Val Val Leu Ala Thr Thr Leu Val Ser
atc cct tca gat cgt gca tct gat ttc agg aat gcc gca gtc cac gag
Ile Pro Ser Asp Arg Ala Ser Asp Phe Arg Asn Ala Ala Val His Glu
aga cag aag gag ctg gtc gtt acg gcc acc acg act tgc tgt ggt tat
Arg Gln Lys Glu Leu Val Val Thr Ala Thr Thr Thr Cys Cys Gly Tyr
aat ccg atg aca tcg tgc cct cgt tgc atg tgc gat agt agc tgc aac
Asn Pro Met Thr Ser Cys Pro Arg Cys Met Cys Asp Ser Ser Cys Asn
aag aaa aaa cca ggc cgc aga aac gac tgatgctcca ggaccctctg
Lys Lys Lys Pro Gly Arg Arg Asn Asp
aaccacgacg t

TABLE 7

DNA (SEQ ID NO:60) and Protein (SEQ ID NO:61)
Sequence of κA A10.2

atg ttc acc gtg ttt ctg ttg gtt gtc ttg gca acc act ctc gtt tcc
Met Phe Thr Val Phe Leu Leu Val Val Leu Ala Thr Thr Leu Val Ser
atc cct tca gat cgt gca tct gat ggc agg aat gcc gta gtc cac gag
Ile Pro Ser Asp Arg Ala Ser Asp Gly Arg Asn Ala Val Val His Glu
aga cag cct tgg ctg gtc cct tcg aaa atc acg aat tgc tgt ggt tat
Arg Gln Pro Trp Leu Val Pro Ser Lys Ile Thr Asn Cys Cys Gly Tyr
aat aac atg gaa atg tgc cct act tgc atg tgc act tat tcc tgt cgc
Asn Asn Met Glu Met Cys Pro Thr Cys Met Cys Thr Tyr Ser Cys Arg
ccc aaa aag aaa aaa cca ggc cac aga aac gac tgatgctcca ggaccctctg
Pro Lys Lys Lys Lys Pro Gly His Arg Asn Asp
aaccacgacg t

TABLE 8

DNA (SEQ ID NO:62) and Protein (SEQ ID NO:63)
Sequence of κA C10.1a

atg ttc acc gtg ttt ctg ttg gtt ggc ttg gca acc act ctc gtt tcc
Met Phe Thr Val Phe Leu Leu Val Gly Leu Ala Thr Thr Leu Val Ser
att cct tca gat ggt gca tct gat ggc aag aat gcc gca gtc cac gag
Ile Pro Ser Asp Gly Ala Ser Asp Gly Lys Asn Ala Ala Val His Glu
aga cag aag gag ctg gtc cct tcg aca atc acg act tgc tgt ggt aat
Arg Gln Lys Glu Leu Val Pro Ser Thr Ile Thr Thr Cys Cys Gly Asn
gaa ccg ggg aca atg tgc cct aaa tgc atg tgc gat aat acc tgt ccc
Glu Pro Gly Thr Met Cys Pro Lys Cys Met Cys Asp Asn Thr Cys Pro
ccc aaa aag aag aaa aga cca ggc cgc aga aac gac tgatgctcca
Pro Lys Lys Lys Lys Arg Pro Gly Arg Arg Asn Asp
ggaccctctg aaccacgacg t

TABLE 9

DNA (SEQ ID NO:64) and Protein (SEQ ID NO:65)
Sequence of κA C10.1b

atg ttc acc gtg ttt ctg ttg gtt ggc ttg gca acc act ctc gtt tcc
Met Phe Thr Val Phe Leu Leu Val Gly Leu Ala Thr Thr Leu Val Ser
att cct tca gat ggt gca tct gat ggc aag aat gcc gca gtc cac gag
Ile Pro Ser Asp Gly Ala Ser Asp Gly Lys Asn Ala Ala Val His Glu
aga cag aag gag ctg gtc cct tcg aca atc acg act tgc tgt ggt cat
Arg Gln Lys Glu Leu Val Pro Ser Thr Ile Thr Thr Cys Cys Gly His
gaa ccg ggg aca atg tgc cct aaa tgc atg tgc gat aat acc tgt ccc
Glu Pro Gly Thr Met Cys Pro Lys Cys Met Cys Asp Asn Thr Cys Pro
ccc aaa aag aag aaa aga cca ggc cgc aga aac gac tgatgctcca
Pro Lys Lys Lys Lys Arg Pro Gly Arg Arg Asn Asp
ggaccctctg aaccacgacg t

TABLE 10

DNA (SEQ ID NO:66) and Protein (SEQ ID NO:67)
Sequence of κA C10.2

atg ttc acc gtg ttt ctg ttg gtt ggc ttg gca acc act ctc gtt tcc
Met Phe Thr Val Phe Leu Leu Val Gly Leu Ala Thr Thr Leu Val Ser
att cct tca gat ggt gca tct gat gtc agg aat gcc gca gtc ctc gag
Ile Pro Ser Asp Gly Ala Ser Asp Val Arg Asn Ala Ala Val Leu Glu
aga cag aag gag ctg gtc gtt acg gcc acc acg act tgc tgt ggt tat
Arg Gln Lys Glu Leu Val Val Thr Ala Thr Thr Thr Cys Cys Gly Tyr
aat ccg atg tca atg tgc cct aaa tgc atg tgc act tat tcc tgt ccc
Asn Pro Met Ser Met Cys Pro Lys Cys Met Cys Thr Tyr Ser Cys Pro
cac caa aag aag aaa aga cca ggc cgc aga aac gao tgatgctcca
His Gln Lys Lys Lys Arg Pro Gly Arg Arg Asn Asp
ggaccctctg aaccacgacg t

TABLE 11

DNA (SEQ ID NO:68) and Protein (SEQ ID NO:69)
Sequence of κA Cr10.1

atg ttc acc gtg ttt ctg ttg gtt gtc ttg gca acc act gtc gtt tcc
Met Phe Thr Val Phe Leu Leu Val Val Leu Ala Thr Thr Val Val Ser
atc cct tca gat cgt gca tct gat ggc agg aat gcc gca gtc aac gag
Ile Pro Ser Asp Arg Ala Ser Asp Gly Arg Asn Ala Ala Val Asn Glu
aga caa cct tgg ctg gtc cct tcg aca atc acg act tgc tgt gga tat
Arg Gln Pro Trp Leu Val Pro Ser Thr Ile Thr Thr Cys Cys Gly Tyr
gat ccg ggg aca aag tgc cct cct tgc agg tgc aat aat acc tgt aaa
Asp Pro Gly Thr Lys Cys Pro Pro Cys Arg Cys Asn Asn Thr Cys Lys
cca aaa aaa cca aaa cca gga aaa ggc cgc aga aac gac tgatgctcca
Pro Lys Lys Pro Lys Pro Gly Lys Gly Arg Arg Asn Asp
ggaccctctg aaccacgacg

TABLE 12

DNA (SEQ ID NO:70) and Protein (SEQ ID NO:71)
Sequence of κA Cn10.1

atg ttc acc gtg ttt ctg ttg gtt gtc ttg gca acc act gtc gtt tcc
Met Phe Thr Val Phe Leu Leu Val Val Leu Ala Thr Thr Val Val Ser
atc cct tca gat cgt gca tct gat ggc agg aat gcc gca gtc cat gag
Ile Pro Ser Asp Arg Ala Ser Asp Gly Arg Asn Ala Ala Val His Glu
aga gcg cct tgg ctg gtc cct tcg caa atc acg act tgc tgt ggt tat
Arg Ala Pro Trp Leu Val Pro Ser Gln Ile Thr Thr Cys Cys Gly Tyr
aat ccg ggg aca atg tgc cct tct tgc atg tgc act aat tcc tgc
Asn Pro Gly Thr Met Cys Pro Ser Cys Met Cys Thr Asn Ser Cys
taaaaaaaaa tggctgatgc tcctggaccc tctgaaccac gacgt

TABLE 13

DNA (SEQ ID NO:72) and Protein (SEQ ID NO:73)
Sequence of κA Cn10.2

atg ttc acc gtg ttt ctg ttg gtt gtc ttg gca acc act gtc gtt tcc
Met Phe Thr Val Phe Leu Leu Val Val Leu Ala Thr Thr Val Val Ser
atc cct tca gat cgt gca tct gat gtc agg aat gcc gca gtc cac gag
Ile Pro Ser Asp Arg Ala Ser Asp Val Arg Asn Ala Ala Val His Glu
aga cag aag gat ctg gtc gtt acg gcc acc acg act tgc tgt ggt tat
Arg Gln Lys Asp Leu Val Val Thr Ala Thr Thr Thr Cys Cys Gly Tyr
aat ccg atg aca ata tgc cct cct tgc atg tgc act tat tcc tgt ccc
Asn Pro Met Thr Ile Cys Pro Pro Cys Met Cys Thr Tyr Ser Cys Pro
ccc aaa aag aaa aaa cca ggc cgc aga aac gac tgatgctcca ggaccctctg
Pro Lys Lys Lys Lys Pro Gly Arg Arg Asn Asp
aaccacgacg t

TABLE 14

DNA (SEQ ID NO:74) and Protein (SEQ ID NO:75)
Sequence of κA M10.2

atg ttc acc gtg ttt ctg ttg gtt gtc ttg gca acc act gtc gtt tcc
Met Phe Thr Val Phe Leu Leu Val Val Leu Ala Thr Thr Val Val Ser
atc cct tca gat cgt gca tct gat ggc agg aat gcc gta gtc cac gag
Ile Pro Ser Asp Arg Ala Ser Asp Gly Arg Asn Ala Val Val His Glu
aga gcg cct gag ctg gtc gtt acg gcc acc acg act tgc tgt ggt ttt
Arg Ala Pro Glu Leu Val Val Thr Ala Thr Thr Thr Cys Cys Gly Phe
gat ccg atg aca tgg tgc cct cct tgc atg tgc act tat tcc tgt tcc
Asp Pro Met Thr Trp Cys Pro Pro Cys Met Cys Thr Tyr Ser Cys Ser
cac caa agg aaa aaa cca ggc cgc aga aac gac tgatgctcca ggaccctctg
His Gln Arg Lys Lys Pro Gly Arg Arg Asn Asp
aaccacgacg t

TABLE 15

DNA (SEQ ID NO:76) and Protein (SEQ ID NO:77)
Sequence of κA U006

atg ttc acc gtg ttt ctg ttg gtt gtc ttg gca acc act gtc gtt tcc
Met Phe Thr Val Phe Leu Leu Val Val Leu Ala Thr Thr Val Val Ser
atc cct tca gat cgt gca tct gat ggc agg aat gcc gta gtc cac gag
Ile Pro Ser Asp Arg Ala Ser Asp Gly Arg Asn Ala Val Val His Glu
aga gcg cct gag ctg gtc gtt acg gcc acc acg aat tgc tgt ggt tat
Arg Ala Pro Glu Leu Val Val Thr Ala Thr Thr Asn Cys Cys Gly Tyr
aat ccg atg aca ata tgc cct cct tgc atg tgc act tat tcc tgt cca
Asn Pro Met Thr Ile Cys Pro Pro Cys Met Cys Thr Tyr Ser Cys Pro
cca aaa aga aaa cca ggc cgc aga aac gac tgatgctcca ggaccctctg
Pro Lys Arg Lys Pro Gly Arg Arg Asn Asp
aaccacgacg ttcgagca

TABLE 16

DNA (SEQ ID NO:78) and Protein (SEQ ID NO:79)
Sequence of κA Mn10.1

atg ttc acc gtg ttt ctg ttg gtt gtc ttg gca acc act ctc gtt tcc
Met Phe Thr Val Phe Leu Leu Val Val Leu Ala Thr Thr Leu Val Ser
atc cct tca gat cgt gca tct gat ttc agg aat gcc gca gtc cac gag
Ile Pro Ser Asp Arg Ala Ser Asp Phe Arg Asn Ala Ala Val His Glu
aga cag aag gag ctg gtc gtt acg gcc acc acg act tgc tgt ggt tat
Arg Gln Lys Glu Leu Val Val Thr Ala Thr Thr Thr Cys Cys Gly Tyr
aat ccg atg aca tcg tgc cct cgt tgc atg tgc gat agt agc tgc aac
Asn Pro Met Thr Ser Cys Pro Arg Cys Met Cys Asp Ser Ser Cys Asn
aag aaa aaa cca ggc cgc aga aac gac tgatgctcca ggaccctctg
Lys Lys Lys Pro Gly Arg Arg Asn Asp
aaccacgacg t

TABLE 17

DNA (SEQ ID NO:80) and Protein (SEQ ID NO:81)
Sequence of κA Mn10.2

atg ttc acc gtg ttt ccg ttg gtc gtc ttg gca acc act ctc gtt tcc
Met Phe Thr Val Phe Pro Leu Val Val Leu Ala Thr Thr Leu Val Ser
atc cct tca gat cgt gca tct gat ggc agg aat gcc gta gtc cac gag
Ile Pro Ser Asp Arg Ala Ser Asp Gly Arg Asn Ala Val Val His Glu
aga cag cct tgg ctg gtc cct tcg aaa atc acg aat tgc tgt ggt tat
Arg Gln Pro Trp Leu Val Pro Ser Lys Ile Thr Asn Cys Cys Gly Tyr
aat acg atg gaa atg tgc cct act tgc atg tgc act tat tcc tgt cgc
Asn Thr Met Glu Met Cys Pro Thr Cys Met Cys Thr Tyr Ser Cys Arg
ccc aaa aag aaa aaa cca ggc cgc aga aac gac tgatgctcca ggaccctctg
Pro Lys Lys Lys Lys Pro Gly Arg Arg Asn Asp
aaccacgacg t

TABLE 18

DNA (SEQ ID NO:82) and Protein (SEQ ID NO:83)
Sequence of κA Sm10.2

atg ttc acc gtg ttt ctg ttg gtt gtc ttg gca acc act gtc gtt tcc
Met Phe Thr Val Phe Leu Leu Val Val Leu Ala Thr Thr Val Val Ser
atc cct tca gat cgt gca tct gat ggc agg aat gcc gca gtc aac gag
Ile Pro Ser Asp Arg Ala Ser Asp Gly Arg Asn Ala Ala Val Asn Glu
aga gcg cct tgg ctg gtc cct tcg aca atc acg act tgc tgt gga tat
Arg Ala Pro Trp Leu Val Pro Ser Thr Ile Thr Thr Cys Cys Gly Tyr
gat ccg ggg aca atg tgc cct cct tgc atg tgc aat aat acc tgt aaa
Asp Pro Gly Thr Met Cys Pro Pro Cys Met Cys Asn Asn Thr Cys Lys
cca aca aaa aaa aga cca ggc cgc aga aac gac tgatgctccc aggaccctct
Pro Thr Lys Lys Arg Pro Gly Arg Arg Asn Asp
gaaccacgac g

TABLE 19

DNA (SEQ ID NO:84) and Protein (SEQ ID NO:85)
Sequence of κA SmVIII

atg ttc acc gtg ttt ctg ttg gtt gtc ttg gca acc act gtc gtt tcc
Met Phe Thr Val Phe Leu Leu Val Val Leu Ala Thr Thr Val Val Ser
atc cct tca gat cgt gca tct gat ggc agg aat gcc gca gtc aac gag
Ile Pro Ser Asp Arg Ala Ser Asp Gly Arg Asn Ala Ala Val Asn Glu
aga caa act tgg ctg gtc cct tcg aca atc acg act tgc tgt gga tat
Arg Gln Thr Trp Leu Val Pro Ser Thr Ile Thr Thr Cys Cys Gly Tyr
gat ccg ggg aca atg tgc cct act tgc atg tgc gat aat acc tgt aaa
Asp Pro Gly Thr Met Cys Pro Thr Cys Met Cys Asp Asn Thr Cys Lys
cca aaa ccc aaa aaa tca ggc cgc aga aac gac tgatgctcca ggaccctctg
Pro Lys Pro Lys Lys Ser Gly Arg Arg Asn Asp
aaccacgacg t

TABLE 20

DNA (SEQ ID NO:86) and Protein (SEQ ID NO:87)
Sequence of κA SmVIIIA

atg ttc acc gtg ttt ctg ttg gtt gtc ttg gca acc act gtc gtt tcc
Met Phe Thr Val Phe Leu Leu Val Val Leu Ala Thr Thr Val Val Ser
atc cct tca gat cgt gca tct gat ggc agg aat gcc gaa gtc aac gag
Ile Pro Ser Asp Arg Ala Ser Asp Gly Arg Asn Ala Glu Val Asn Glu
aga gcg cct tgg ctg gtc cct tcg aca atc acg act tgc tgt gga tat
Arg Ala Pro Trp Leu Val Pro Ser Thr Ile Thr Thr Cys Cys Gly Tyr
gat ccg ggg tca atg tgc cct cct tgc atg tgc aat aat acc tgt aaa
Asp Pro Gly Ser Met Cys Pro Pro Cys Met Cys Asn Asn Thr Cys Lys
cca aaa ccc aaa aaa tca ggc cgc aga aac cac tgatgctcca ggaccctctg
Pro Lys Pro Lys Lys Ser Gly Arg Arg Asn His
aaccacgacg ttcgagca

TABLE 21
DNA (SEQ ID NO:88) and Protein (SEQ ID NO:89) Sequence of κA SIVA

atg ttc acc gtg ttt ctg ttg gtt gtc ttg gca acc aat gtc gtt tcc
Met Phe Thr Val Phe Leu Leu Val Val Leu Ala Thr Asn Val Val Ser
acc cct tca gat cgt gca tct gat ggc agg aat gcc gca gtc cac gag
Thr Pro Ser Asp Arg Ala Ser Asp Gly Arg Asn Ala Ala Val His Glu
aga cag aag agt ctg gtc cct tcg gtc atc acg act tgc tgt gga tat
Arg Gln Lys Ser Leu Val Pro Ser Val Ile Thr Thr Cys Cys Gly Tyr
gat ccg ggg aca atg tgc cct cct tgc agg tgc act aat agc tgt ggt
Asp Pro Gly Thr Met Cys Pro Pro Cys Arg Cys Thr Asn Ser Cys Gly
taaccaaaac ccaaaacagg ccgcagaaac gactgatgct ccaggaccct ctgaaccacg
acgttcgagc a

TABLE 22

DNA (SEQ ID NO:90) and Protein (SEQ ID NO:91)
Sequence of κA SVIIIA

atg ttc acc gtg ttt ctg tcg gtt gtc ttg gca acc act gtc gtt tcc
Met Phe Thr Val Phe Leu Ser Val Val Leu Ala Thr Thr Val Val Ser
acc cct tca gat cgt gca tct gat ggc agg aat gcc gca gtc cac gag
Thr Pro Ser Asp Arg Ala Ser Asp Gly Arg Asn Ala Ala Val His Glu
aga cag aag gag ctg gtc cct tcg gtc atc acg act tgc tgt gga tat
Arg Gln Lys Glu Leu Val Pro Ser Val Ile Thr Thr Cys Cys Gly Tyr
gat ccg ggg aca atg tgc cct cct tgc agg tgc act aat tcc tgt cca
Asp Pro Gly Thr Met Cys Pro Pro Cys Arg Cys Thr Asn Ser Cys Pro
aca aaa ccg aaa aaa cca ggc cgc aga aac gac tgatgctcca ggaccctctg
Thr Lys Pro Lys Lys Pro Gly Arg Arg Asn Asp
aaccacgacg ttcgagca

TABLE 23

DNA (SEQ ID NO:92) and Protein (SEQ ID NO:93)
Sequence of κA Sx10.1

atg ttc acc gtg ttt ctg ttg gtt gtc ttg gca acc act gtc gtt tcc
Met Phe Thr Val Phe Leu Leu Val Val Leu Ala Thr Thr Val Val Ser
atc cct tca gat cgt gca tat gat ggc aag aat gcc gca gtc cac gag
Ile Pro Ser Asp Arg Ala Tyr Asp Gly Lys Asn Ala Ala Val His Glu
aga caa tct tgg ctg gtc cct tcg aca atc acg act tgc tgt ggt tat
Arg Gln Ser Trp Leu Val Pro Ser Thr Ile Thr Thr Cys Cys Gly Tyr
agt ccg ggg aca atg tgc cct cct tgc atg tgc act aat acc tgc
Ser Pro Gly Thr Met Cys Pro Pro Cys Met Cys Thr Asn Thr Cys
taaaaaaatg gctgatgctc caggaccctc tgaaccacga cgt

TABLE 24

DNA (SEQ ID NO:94) and Protein (SEQ ID NO:95)
Sequence of κA S110.1

atg ttc acc gtg ttt ctg ttg gtt gtc ttg gca acc act gtc gtt tcc
Met Phe Thr Val Phe Leu Leu Val Val Leu Ala Thr Thr Val Val Ser
atc cct tca gat cgt gca tct gat gtc agg aat gcc gca gtc cac gag
Ile Pro Ser Asp Arg Ala Ser Asp Val Arg Asn Ala Ala Val His Glu
aga cag aag gat ctg gtc gtt acg gcc acc acg act tgc tgt ggt tat
Arg Gln Lys Asp Leu Val Val Thr Ala Thr Thr Thr Cys Cys Gly Tyr
aat ccg atg aca atg tgc cct cct tgc atg cgc act tat tcc tgt tcc
Asn Pro Met Thr Met Cys Pro Pro Cys Met Arg Thr Tyr Ser Cys Ser
ccc aaa aag aaa aaa cca ggc cgc aga aac gac tgatgctcca ggaccctctg
Pro Lys Lys Lys Lys Pro Gly Arg Arg Asn Asp
aaccacgacg t

TABLE 25
DNA (SEQ ID NO:96) and Protein (SEQ ID NO:97)
Sequence of κA S110.2

atg ttc acc gtg ttt ctg ttg gtt gtc ttg gca acc act gtc gtt tcc
Met Phe Thr Val Phe Leu Leu Val Val Leu Ala Thr Thr Val Val Ser
acc cct tca gat cgt gca tct gat ggc agg aat gcc gca gtc cac ggg
Thr Pro Ser Asp Arg Ala Ser Asp Gly Arg Asn Ala Ala Val His Gly
aga cag aag gag ctg gtc cct tcg gtc atc acg act tgc tgt gga tat
Arg Gln Lys Glu Leu Val Pro Ser Val Ile Thr Thr Cys Cys Gly Tyr
gat ccg ggg aca atg tgc cct cct tgc agg tgc act aat tcc tgt cca
Asp Pro Gly Thr Met Cys Pro Pro Cys Arg Cys Thr Asn Ser Cys Pro
aca aaa ccg aaa aaa cca ggc cgc aga aac gac tgatgctcca ggaccctctg
Thr Lys Pro Lys Lys Pro Gly Arg Arg Asn Asp
aaccacgacg t

### Example 7

### Preparation of κA A671

Synthesis. The linear κA peptide A671 was synthesized on a 35 7ACT peptide synthesizer (Advanced Chemtech, Louisville, Ky.) using a Fmoc-chemistry strategy on a Rink amide MBHA resin. For this peptide, all Cys residues were protected as the acid-labile Cys(S-trityl). Side-chain protection of non-Cys residues was in the form of trityl (Asn), t-butyloxycarbonyl (Trp), t-butyl (Asp, Ser, Thr, Tyr) and pentamethylchromansulfonyl (Arg). Following synthesis, the terminal Fmoc group was removed with 20% piperidine in dimethylformamide. Linear peptide was cleaved from the solid support by treatment with trifluoroacetic acid/phenol/ethanedithiol/thioanisole (90/5/2.5/2.5 by volume). This procedure cleaved the peptide from the resin and deprotected the Cys (S-trityl) and the non-Cys residue side chains. Cleavage mixture was vacuum filtered through a fritted syringe to remove resin. Cleavage vessel was also rinsed with TFA and filtered. The peptide was precipitated by addition of methyl-t-butyl ether (MTBE) chilled to −20° C. The precipitate was washed four additional times with cold MTBE and the supernatants were discarded. The inear peptide was then lyophilized and stored at −80° C.

Folding. Glutathiole (GSSG/GSH) oxidation is used to form the three disulfide bridges. Peptide is dissolved in 40% acetonitrile (ACN) and water. Stock solution of GSSG/GSH (20 M/40 mM) is prepared. GSH stock solution is added to the peptide solution to make a final concentration of 0.5 mM GSSG/1.0 mM GSH. The pH is adjusted to 7.5-8.0 with Na₂HPO₄ (0.25 M). Solution is covered at room temperature overnight. The peptide solution is acidified to pH 5 with 50% acetic acid. Peptide is then analyzed by HPLC to check yield and purity before preparative HPLC. The solution is diluted three times by volume with H₂O and purified by RP-HPLC.

RP-HPLC. Preparative purification was done on a Waters Prep LC 4000 with a Waters 2487 detector (Waters Corp., Milford, Mass.). Analytical HPLC consisted of Dynamax pumps and a Dynamax UVDII detector (Varian/Rainin, Woburn, Mass.). Peptide purification was done on a preparative Vydac C18 column (22mm×25cm, 10 μm particle size, 300 Å pore size). All other analytical HPLC was done on an analytical Vydac C18 column (4.6mm×25cm, 5 μm particle size, 300 Å pore size). For prep and analytical HPLC, buffer A was 0.1% TFA in H₂O and buffer B was 0.085% TFA, 90% acetonitrile in H₂O.

After folding of the reduced A671, analytical HPLC showed the presence of two major products (A671-peak 1 and A671-peak 2). These two folding products were separated easily by HPLC. Mass spectrometry results were obtained: A671-peak 1, 3247.68±0.73 and A671-peak 2, 3247.94±0.35. The results show that the two peaks are different folding isomers of the peptide A671.

### Example 8

### Materials and Methods for κA A671 Activity Analysis

Primary cultures of rat cortex. Neonatal rats were killed by decapitation. The cortical hemispheres were removed, cleaned of meninges and the hippocampus removed and discarded. The cortex was dissociated using 20 U/ml Papain with constant mixing for 45 min at 37° C. Digestion was terminated with fraction V BSA (1.5 mg/ml) and Trypsin inhibitor (1.5 mg/ml) in 10 mls media (DMEM/F12±10% fetal Bovine serum±B27 neuronal supplement; Life Technologies). Using gentle trituration cells were separated from the surrounding connective tissue. Using a fluid-handling robot (Quadra 96, Tomtec) cells were settled onto uncoated coverslips or Primaria-treated 96 well plates (Becton-Dickenson). Each well was loaded with approximately 25,000 cells. Plates and coverslips were placed into a humidified 5% CO₂ incubator at 37° C. and kept for at least 5 days before fluorescence screening.

The saline solution contained (in mM) 137 NaCl, 5 KCl, 10 HEPES, 25 Glucose, 3 CaCl₂, and 1 MgCl₂ (brought to pH 7.3 with NaOH).

96 well plate fluorimetry protocol. Prior to beginning the experiments the cells were washed thoroughly with saline solution The Fluo-3 calcium dye was loaded into the cytoplasm with 20% pluronic acid where esterases cleave the dye from the ester effectively trapping the dye within the cell. Increases in intracellular calcium measured with the Fluo-3 dye are reflected as rises in fluorescence and decreases reflect a drop in fluorescence.

Guide to Interpreting Fluorimetry. Fluorometric measurements of a mixed cortical preparation are an averaging of cellular responses from approximately 25,000 cells per well of a 96 well plate. Cultures of cells from the cortex include at least pyramidal neurons, bipolar neurons, intemeurons and astrocytes. Changes in intracellular Ca²⁺ (Fluo-3) were used as a measure of the response elicited with κA A671 alone or with κA A671 in the presence of specific receptor/ion channel agonists or antagonists. Cultures are effected by lenght of time in vitro, extracellular matirx and saline conditions. In order to minimize well-to-well variability, each well acted as its own control by comparing the degree of fluorescence in pretreatment to that in post-treatment. This normalization process allows comparison of relative responses from plate to plate and culture to culture. Mixed-cell populations in each well were measured with the fluorimeter, and individual cell signaling responses were averaged. Statistics, including mean and standard error of the mean, from eight wells allowed for comparison of significant differences between treatments. Results were expressed as percent change in fluorescence.

### Example 9

### κA A671 Increases Intracellular Calcium in Depolarized Preparations

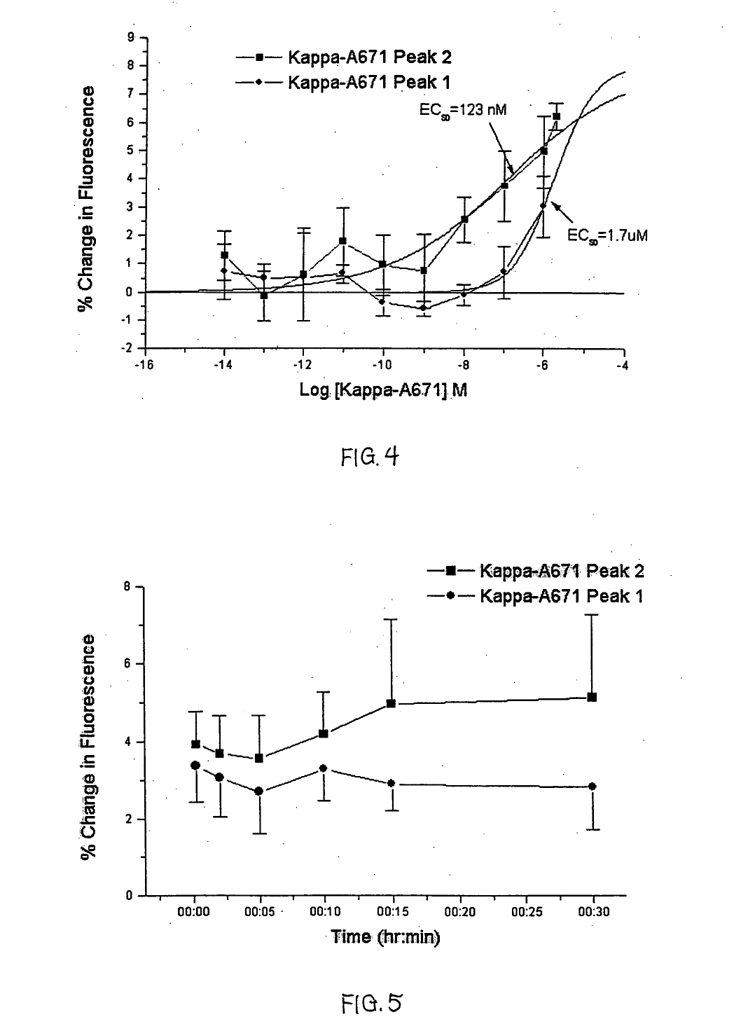

Primary cultures of neonatal rat cortex were depolarized by pretreating with 1-10 uM Aconitine (a sodium channel activator). This depolarization results in a sustained influx of calcium ions through the activation of voltage-gated calcium channels. In the continued presence of aconitine increasing concentrations of the synthesized Conus peptide kappa-A A671 (both peaks 1 and 2) produced a further significant enhancement of the intracellular calcium levels (FIG. 4) measured with the calcium dye Fluo-3. κA A671 peak 2 showed approximately 10-fold greater potency than peak 1 (123 nM vs 1.7 uM respectively).

No significant changes were detected in the κA A671 induced response over time (up to 30min, FIG. 5). The first fluorimetric recording was taken at 15 sec so any fast and inactivating events could not be resolved using this method.

### Example 10

### κA A671 Induced Increase in Calcium is 4-Aminopyridine Sensitive

It is possible that the κA A671 induced increase in calcium could be due to a blockade of voltage-gated K+ channels. Under depolarized conditions an inhibition of these channels would result in a reduction in K+ efflux and an enhancement of the calcium influx. To examine this further the ability of kappa-A A671 to compete with a general antagonist of the voltage-gated K+ channels, 4-aminopyridine (4-AP), was assessed. If the 4-AP and the κA A671 were acting through independent mechanisms their effects should be additive and in the presence of the 4-AP the kappa-A A671 should be able to produce an increase in calcium. If they were acting through the same mechanism (directly or indirectly) then the response to κA A671 would be reduced in a dose-dependent manner by the 4-AP pretreatment.

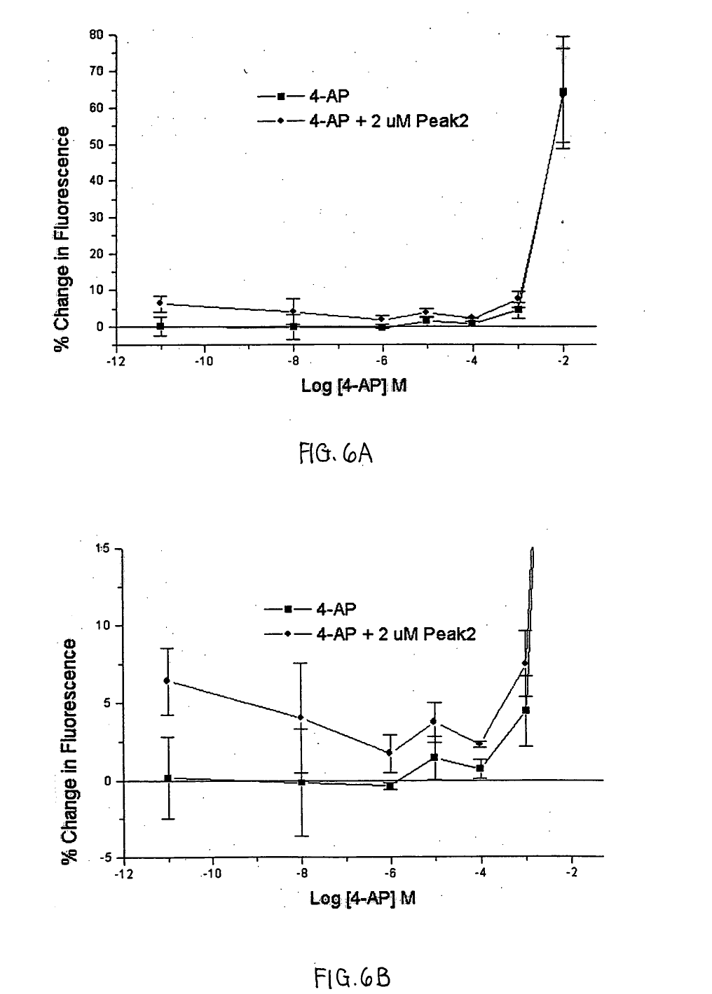

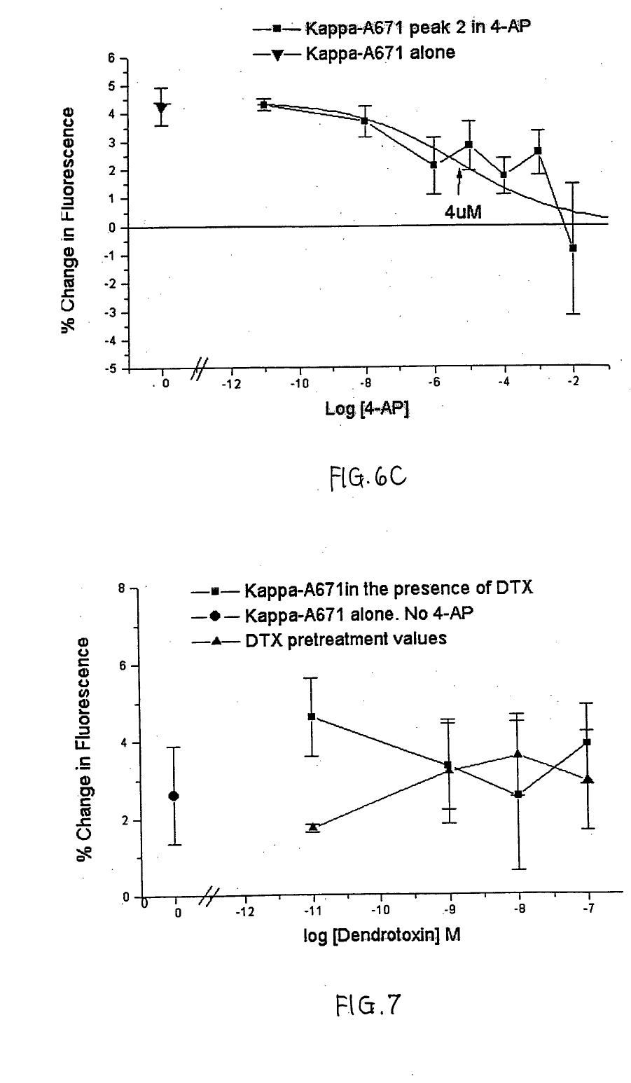

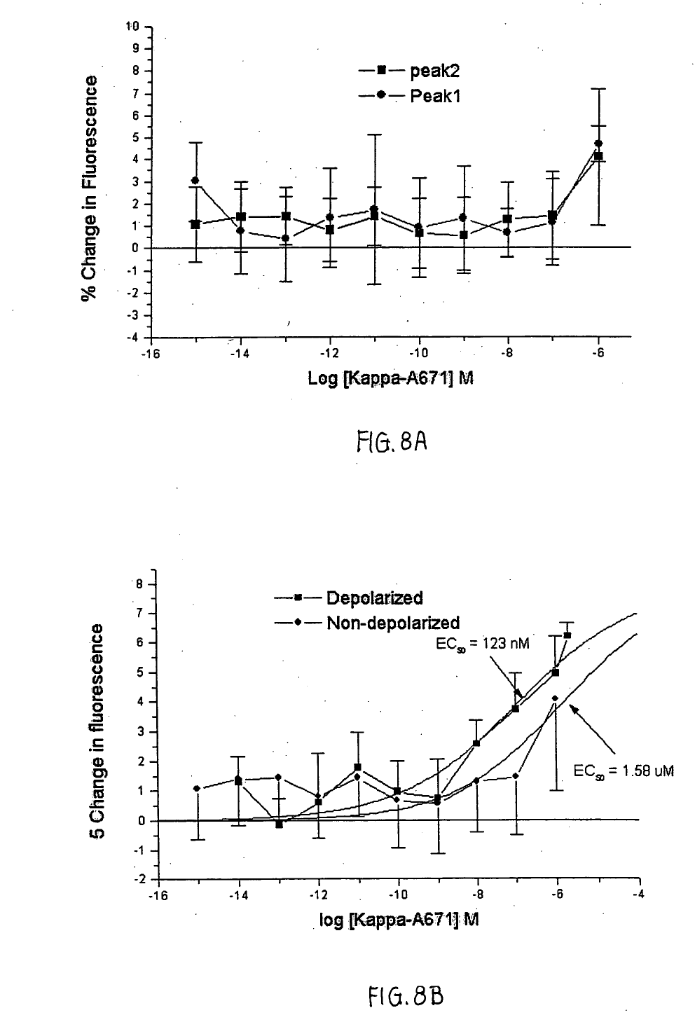

Initially voltage gated K⁺ channels were blocked by pretreating the cells with 4-AP in the presence of aconitine (FIG. 6A). The cells were then exposed to κA A671 (2 uM peak2) in the continued presence of the 4-AP. As can be seen from FIGS. 6B and 6C, this 4-AP pretreatment reduced the size of the κA A671 (Peak 2) induced response. This effect was dose dependent.

### Example 11

### κA A671 Induced Increase in Calcium is Unaffected by Dendrotoxin

Dendrotoxin (DTX) is a peptide isolated from the venom of the green mamba that specifically targets the Kv1.1, Kv1.2 and Kv1.6 voltage-gated K channels. To evaluate the involvement of these channels in the κA A671 response the ability of DTX to compete with κA A671 was examined as outlined above for 4-AP. Under depolarized conditions pretreatment with dendrotoxin caused an increase in intracellular calcium (FIG. 7). In the continued presence of the dendrotoxin peak 2 of the κA A671 was still able to produce a significant increase in the intracellular calcium. This suggests that the above mentioned channels are not involved in the κA A671 induced response.

### Example 12

### κA A671 Is Also Active in a Non-Depolarized Environment

In preparations not treated with Aconitine, both peaks of κA A671 still produced significant increases in intracellular calcium (FIG. 8) at 1 uM. As can be seen from FIG. 8B, the potency of κA A671 peak 2 is reduced slightly in the non-depolarized environment compared to the depolarized environment (1.58 uM vs. 123 nM respectively. Peak 1 appears to be equally potent in both depolarized and non-depolarized cultures (FIG. 8C).

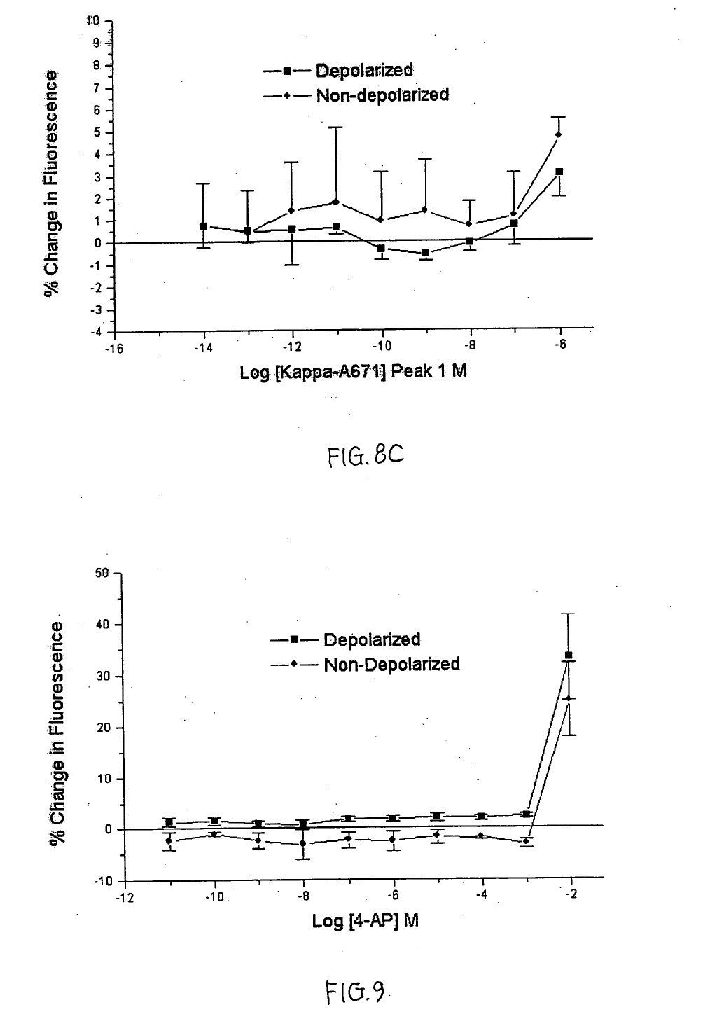

If κA A671 acts through a blockade of voltage-gated K+ channels no activity would be expected in non-depolarized preparations where it would be anticipated that the channels would be closed. One possibility for the activity seen is that the preparations at “rest” are somewhat depolarized (perhaps as a result of spontaneous neurotransmitter release). As such the effect of 4-AP was examined under the same conditions. Here, too, we can see that there is a significant effect of the compound without pretreatment with a depolarizing agent (FIG. 9). This effect was slightly less potent in the non-depolarized state vs. the cells that had been pretreated with Aconitine. This confirms the depolarized nature of primary cultures of cortical cells even in the absence of a specific depolarizing agent.

The following conclusions can be drawn from Examples 8-12. (1) Both Peaks of the κA A671 peptide produced significant dose-dependent increases in intracellular calcium in depolarized preparations. (2) Using the fluorimetric assay Peak 2 appears to be the more potent of the 2 peaks with an EC₅₀ of 123 nM compared to 1.7 uM derived from Peak 1. (3) The response induced byPeak 2 could be inhibited by pretreating with the voltage-gated K⁺ channel blocker 4-AP indicating that Peak 2 actively blocks 4-AP sensitive K⁺ channels. The response however was unaffected by the presence of dendrotoxin indicating that the Kv1.1, Kv1.2 and Kv1.6 voltage gated K channels were not involved in this process. (4) Both Peaks were also active in cells not pretreated with a depolarizing agent although peak 2 appears slightly less potent in the non-depolarized environment. This activity is probably a result of the cells being somewhat depolarized. This was confirmed with the finding that 4-AP is also active in untreated preparations.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

LIST OF REFERENCES

Barber, M. et al. (1982). Anal. Chem. 54:645A-657A.
Bodanskyet al. (1966). Chem. Ind. 38:1597-98.
Cartier, G. E. et al. (1996). J. Biol. Chem. 271:7522-7528.
Cruz, L. J. et al. (1987). J. Biol. Chem. 260:9280-9288.
Dodd, J. et al. (1983). J. Physiol. 334:255-268.
Gobbo, M. et al. (1992). Int. J. Pept. Protein Res. 40:51-54.
Haack, J. A. et al. (1990). J. Biol. Chem. 265:6025-6029.
Hammerland, L. G. et al. (1992). Eur. J. Pharmacol. 226:239-244.
Horiki, K. et al. (1978). Chemistry Letters 165-68.
Kaiser et al. (1970). Anal. Biochem. 34:595.
Kapoor (1970). J. Pharm. Sci. 59:1-27.
Lowry, O. et al. (1951). J. Biol. Chem. 193:265.
Mena, E. E. et al. (1990). Neurosci. Lett. 118:241-244.
Methoden der Organischen Chemie (Houben-Weyl): Synthese von Peptiden, E. Wunsch (Ed.), Georg Thieme Verlag, Stuttgart, Ger. (1974).
Nishiuchi, Y. et al. (1993). Int. J. Pept. Protein Res. 42:533-538.
Olivera, B. M. et al. (1985). Science 230:1338-1343.
Piscano, A. et al. (1993). Glycobiology 3:429-435.
Remington 's Pharmaceutical Sciences, 18th Ed., Mack Publishing Co., Easton, PA (1990).
Rivier, J. R. et al. (1978). Biopolymers 17:1927-38.
Rivier, J. R. et al. (1987). Biochem. 26:8508-8512.
Sambrook, J. et al. (1989). Molecular Cloning: A Laboratory Manual, 2nd Ed., Cold Spring Harbor Laboratory, Cold Spring Harbor, Ny.
Schroder & Lubke (1965). The Peptides 1:72-75, Academic Press, NY.
Shon, K-J. et al. (1998). J. Biol. Chem. 273:33-38.
Stewart and Young, Solid-Phase Peptide Synthesis, Freeman & Co., San Francisco, Calif. (1969).
Van de Steen et al. (1998). Crit. Rev. Biochem. Mol. Biol. 33:151-208.
Yoshikami, D. et al. (1989). Ann. N.Y. Acad. Sci. 560:230-248.
Zhou L. M., et al. (1996). J. Neurochem. 66:620-628.
U.S. application Ser. No. 08/785,534
U.S. application Ser. No. 09/061,026
U.S. Pat. No. 3,842,067.
U.S. Pat. No. 3,862,925.
U.S. Pat. No. 3,972,859.
U.S. Pat. No. 4,105,603.
U.S. Pat. No. 4,447,356.
U.S. Pat. No. 4,569,967.
U.S. Pat. No. 5,364,769.
U.S. Pat. No. 5,514,774.
U.S. Pat. No. 5,534,615.
U.S. Pat. No. 5,550,050.
U.S. Pat. No. 5,591,821.
U.S. Pat. No. 5,633,347.
U.S. Pat. No. 5,635,347.
U.S. Pat. No. 5,844,077.
PCT Published Application WO 92/19125.
PCT Published Application WO 94/25503.
PCT Published Application WO 95/01203.
PCT Published Application WO 96/02286.
PCT Published Application WO 96/02646.
PCT Published Application WO 96/05452.
PCT Published Application WO 96/11698.
PCT Published Application WO 96/40871.
PCT Published Application WO 96/40979.
PCT Published Application WO 97/12635.

1. A method for regulating the flow of potassium through potassium channels in an individual in need thereof which comprises administering a therapeutically effective amount of a κA conopeptide.

2. The method of claim 1, wherein said individual in need thereof suffers from a disorder selected from the group consisting of multiple sclerosis, other demyelinating diseases (such as acute dissenmiated encephalomyelitis, optic neuromyelitis, adrenoleukodystrophy, acute transverse myelitis, progressive multifocal leukoencephalopathy), sub-acute sclerosing panencephalomyelitis (SSPE), metachromatic leukodystrophy, Pelizaeus-Merzbacher disease, spinal cord injury, botulinum toxin poisoning, Huntington's chorea, compression and entrapment neurophathies (such as carpal tunnel syndrome, ulnar nerve palsy), cardiovascular disorders (such as cardiac arrhythmias, congestive heart failure), reactive gliosis, hyperglycemia, immunosuppression, cocaine addiction, cancer, cognitive dysfunction, disorders resulting from defects in neurotransmitter release (such as Eaton-Lambert syndrome), and reversal of the actions of curare and other neuromuscular blocking drugs.

3. The method of claim 1, wherein said disorder is a demyelinating disease.

4. The method of claim 1, wherein said κA conopeptide has the general formula:
(SEQ ID NO:43)-Z16
Xaa1-Xaa2-Xaa3-Leu-Val-Xaa4-Xaa5-Xaa6-Xaa7-Thr-
Thr-Cys-Cys-Gly-Xaa8-Xaa9-Xaa4-Xaa10-Xaa5-Xaa11-
Cys-Xaa12-Xaa12-Cys-Xaa13-Cys-Xaa14-Xaa15-Xaa12-
Cys-Cys,
wherein Xaa1 is Ala, Glu, Gln, pyro-Glu or γ-carboxy-Glu, Xaa2 is Pro, hydroxy-Pro, Ser, Thr or Lys, Xaa3 is Trp, D-Trp, bromo-Trp, Glu or γ-carboxy-Glu, Xaa4 is Pro, hydroxy-Pro or Val, Xaa5 is Ser or Thr, Xaa6 is Ala, Thr or Val, Xaa7 is Thr or lie, Xaa8 is Tyr, mono-iodo-Tyr, di-iodo-Tyr, O-sulpho-Tyr, O-phospho-Tyr or nitro-Tyr, Xaa9 is Asp or Asn, Xaa10 is Met or Gly, Xaa11 is Met, Trp, D-Trp, bromo-Trp, Ile, Nle or Leu, Xaa12 is Pro, hydroxy-Pro, Ser or Thr, Xaa13 is Arg or Met, Xaa14 is Asp, Asn, Thr or Ser, Xaa15 is Asn, His or Tyr,mono-iodo-Tyr, di-iodo-Tyr, O-sulpho-Tyr, O-phospho-Tyr or nitro-Tyr and Z16 is des-Z16 or a peptide of the formulaA-B, where A is peptide selected from the group of peptides having SEQ ID NOs:26-38 and B is des-B or a peptide selected from the group of peptides having SEQ ID NOs:39-42.

5. The method of claim 4, wherein said κA conopeptide is further modified to comprise an O-glycan.

6. The method of claim 1, wherein said κA conopeptide has the consensus formula:
(SEQ ID NO:44)
Xaa1-Xaa2-Xaa3-Leu-Val-Xaa4-Ser-Xaa5-Ile-Thr-Thr-
Cys-Cys-Gly-Tyr-Asp-Xaa4-Gly-Thr-Met-Cys-Xaa4-
Xaa4-Cys-Xaa6-Cys-Thr-Asn-Xaa7-Cys
wherein Xaa1 is Ala, Glu, Gln, pyro-Glu or γ-carboxy-Glu, Xaa2 is Pro, hydroxy-Pro, Ser, Thr or Lys, Xaa3 is Trp, D-Trp, bromo-Trp, Glu or γ-carboxy-Glu, Xaa4 is Pro or hydroxy-Pro, Xaa5 is Ala, Thr or Val, Xaa6 is Met or Arg and Xaa7 is Thr or Ser.

7. The method of claim 6, wherein said κA conopeptide is further modified to comprise an O-glycan.

8. The method of claim 1, wherein said κA conopeptide is selected from the group consisting of:
(SEQ ID NO:1)-Z1
κA A10.1: Xaa2-Lys-Xaa1-Leu-Val-Val-Thr-Ala-Thr-
Thr-Thr-Cys-Cys-Gly-Xaa5-Asn-Xaa3-Met-Thr-Ser-
Cys-Xaa3-Arg-Cys-Met-Cys-Asp-Ser-Ser-Cys;
(SEQ ID NO:2)-Z2
κA A10.2: Xaa2-Xaa3-Xaa4-Leu-Val-Xaa3-Ser-Lys-
Ile-Thr-Asn-Cys-Cys-Gly-Xaa5-Asn-Asn-Met-Xaa1-
Met-Cys-Xaa3-Thr-Cys-Met-Cys-Thr-Xaa5-Ser-Cys;
(SEQ ID NO:3)-Z3
κA C10.1a: Xaa2-Lys-Xaa1-Leu-Val-Xaa3-Ser-Thr-
Ile-Thr-Thr-Cys-Cys-Gly-Asn-Xaa1-Xaa3-Gly-Thr-
Met-Cys-Xaa3-Lys-Cys-Met-Cys-Asp-Asn-Thr-Cys;
(SEQ ID NO:4)-Z3
κA C10.1b: Xaa2-Lys-Xaa1-Leu-Val-Xaa3-Ser-Thr-
Ile-Thr-Thr-Cys-Cys-Gly-His-Xaa1-Xaa3-Gly-Thr-
Met-Cys-Xaa3-Lys-Cys-Met-Cys-Asp-Asn-Thr-Cys;
(SEQ ID NO:5)-Z4
κa C10.2: Xaa2-Lys-Xaa1-Leu-Val-Val-Thr-Ala-Thr-
Thr-Thr-Cys-Cys-Gly-Xaa5-Asn-Xaa3-Met-Ser-Met-
Cys-Xaa3-Lys-Cys-Met-Cys-Thr-Xaa5-Ser-Cys;
(SEQ ID NO:6)-Z5
κA Cr10.1: Xaa2-Xaa3-Xaa4-Leu-Val-Xaa3-Ser-Thr-
Ile-Thr-Thr-Cys-Cys-Gly-Xaa5-Asp-Xaa3-Gly-Thr-
Lys-Cys-Xaa3-Xaa3-Cys-Arg-Cys-Asn-Asn-Thr-Cys;
(SEQ ID NO:7)
κa Cn101.1: Ala-Xaa3-Xaa4-Leu-Val-Xaa3-Ser-Gln-
Ile-Thr-Thr-Cys-Cys-Gly-Xaa5-Asn-Xaa3-Gly-Thr-
Met-Cys-Xaa3-Ser-Cys-Met-Cys-Thr-Asn-Ser-Cys;
(SEQ ID NO:8)-Z6
κA Cn10.2: Xaa2-Lys-Asp-Leu-Val-Val-Thr-Ala-Thr-
Thr-Thr-Cys-Cys-Gly-Xaa5-Asn-Xaa3-Met-Thr-Ile-
Cys-Xaa3-Xaa3-Cys-Met-Cys-Thr-Xaa5-Ser-Cys;
(SEQ ID NO:9)-Z7
κA M10.2: Ala-Xaa3-Xaa1-Leu-Val-Val-Thr-Ala-Thr-
Thr-Thr-Cys-Cys-Gly-Phe-Asp-Xaa3-Met-Thr-Xaa4-
Cys-Xaa3-Xaa3-Cys-Met-Cys-Thr-Xaa5-Ser-Cys;
(SEQ ID NO:10)-Z8
κA U006: Ala-Xaa3-Xaa1-Leu-Val-Val-Thr-Ala-Thr-
Thr-Asn-Cys-Cys-Gly-Xaa5-Asn-Xaa3-Met-Thr-Ile-Cys-
Xaa3-Xaa3-Cys-Met-Cys-Thr-Xaa5-Ser-Cys;
(SEQ ID NO:11)-Z1
κA Mn10.1: Xaa2-Lys-Xaa1-Leu-Val-Val-Thr-Ala-Thr-
Thr-Thr-Cys-Cys-Gly-Xaa5-Asn-Xaa3-Met-Thr-Ser-Cys-
Xaa3-Arg-Cys-Met-Cys-Asp-Ser-Ser-Cys;
(SEQ ID NO:12)-Z9
κA Mn10.2: Xaa2-Xaa3-Xaa4-Leu-Val-Xaa3-Ser-Lys-
Ile-Thr-Asn-Cys-Cys-Gly-Xaa5-Asn-Thr-Met-Xaa1-Met-
Cys-Xaa3-Thr-Cys-Met-Cys-Thr-Xaa5-Ser-Cys;
(SEQ ID NO:13)-Z10
κA Sm10.2: Ala-Xaa3-Xaa4-Leu-Val-Xaa3-Ser-Thr-Ile-
Thr-Thr-Cys-Cys-Gly-Xaa5-Asp-Xaa3-Gly-Thr-Met-Cys-
Xaa3-Xaa3-Cys-Met-Cys-Asn-Asn-Thr-Cys;
(SEQ ID NO:14)-Z11
κA Sm10.3: Xaa2-Ala-Xaa3-Leu-Val-Xaa3-Ser-Thr-Ile-
Thr-Thr-Cys-Cys-Gly-Xaa5-Asp-Xaa3-Gly-Thr-Met-Cys-
Xaa3-Thr-Cys-Met-Cys-Asn-Asn-Thr-Cys;
(SEQ ID NO:15)-Z11
κA SmVIII: Xaa2-Thr-Xaa4-Leu-Val-Xaa3-Ser-Thr-Ile-
Thr-Thr-Cys-Cys-Gly-Xaa5-Asp-Xaa3-Gly-Thr-Met-Cys-
Xaa3-Thr-Cys-Met-Cys-Asp-Asn-Thr-Cys;
(SEQ ID NO:16)-Z12
κA SmVIIIA: Ala-Xaa3-Xaa4-Leu-Val-Xaa3-Ser-Thr-
Ile-Thr-Thr-Cys-Cys-Gly-Xaa5-Asp-Xaa3-Gly-Ser-Met-
Cys-Xaa3-Xaa3-Cys-Met-Cys-Asn-Asn-Thr-Cys;
(SEQ ID NO: 17)
κA SIVA: Xaa2-Lys-Ser-Leu-Val-Xaa3-Ser-Val-Ile-
Thr-Thr-Cys-Cys-Gly-Xaa5-Asp-Xaa3-Gly-Thr-Met-Cys-
Xaa3-Xaa3-Cys-Arg-Cys-Thr-Asn-Ser-Cys;
(SEQ ID NO:18)-Z13
κA SVIIIA: Xaa2-Lys-Xaa1-Leu-Val-Xaa3-Ser-Val-Ile-
Thr-Thr-Cys-Cys-Gly-Xaa5-Asp-Xaa3-Gly-Thr-Met-Cys-
Xaa3-Xaa3-Cys-Arg-Cys-Thr-Asn-Ser-Cys;
(SEQ ID NO:19)
κA Sx10.1: Xaa2-Ser-Xaa4-Leu-Val-Xaa3-Ser-Thr-Ile-
Thr-Thr-Cys-Cys-Gly-Xaa5-Ser-Xaa3-Gly-Thr-Met-Cys-
Xaa3-Xaa3-Cys-Met-Cys-Thr-Asn-Thr-Cys;
(SEQ ID NO:20)-Z14
κA Sl10.1: Xaa2-Lys-Asp-Leu-Val-Val-Thr-Ala-Thr-
Thr-Thr-Cys-Cys-Gly-Xaa5-Asn-Xaa3-Met-Thr-Met-Cys-
Xaa3-Xaa3-Cys-Met-Arg-Thr-Xaa5-Ser-Cys;
(SEQ ID NO:21)-Z13
κA S110.2: Xaa2-Lys-Xaa1-Leu-Val-Xaa3-Ser-Val-Ile-
Thr-Thr-Cys-Cys-Gly-Xaa5-Asp-Xaa3-Gly-Thr-Met-Cys-
Xaa3-Xaa3-Cys-Arg-Cys-Thr-Asn-Ser-Cys;
(SEQ ID NO:22)
κA A671: Ala-Xaa3-Xaa4-Leu-Val-Xaa3-Ser-Thr-Ile-
Thr-Thr-Cys-Cys-Gly-Xaa5-Asn-Xaa3-Gly-Thr-Met-Cys-
Xaa3-Xaa3-Cys-Arg-Cys-Asp-Asn-Thr-Cys;
(SEQ ID NO:23)-Z5
κA H350: Xaa2-Ser-Xaa4-Leu-Val-Xaa3-Ser-Thr-Ile-
Thr-Thr-Cys-Cys-Gly-Xaa5-Asp-Xaa3-Gly-Thr-Met-
Cys-Xaa3-Xaa3-Cys-Arg-Cys-Asn-Asn-Thr-Cys;
(SEQ ID NO:24)-Z8
κA J454: Ala-Xaa3-Xaa1-Leu-Val-Val-Thr-Ala-Thr-
Thr-Thr-Cys-Cys-Gly-Xaa5-Asp-Xaa3-Met-Thr-Ile-Cys-
Xaa3-Xaa3-Cys-Met-Cys-Thr-His-Ser-Cys;
and
(SEQ ID NO:25)-Z15
κA G851: Ala-Xaa3-Xaa1-Leu-Val-Val-Thr-Ala-Thr-
Thr-Thr-Cys-Cys-Gly-Xaa5-Asp-Xaa3-Met-Thr-Xaa4-
Cys-Xaa3-Ser-Cys-Met-Cys-Thr-Xaa5-Ser-Cys,
wherein Xaa1 is Glu or γ-carboxy-Glu, Xaa2 is Gln or pyro-Glu, Xaa3 is Pro or hydroxy-Pro, Xaa4 is Trp, D-Trp orbromo-Trp, Xaa5 is Tyr, mono-iodo-Tyr, di-iodo-tyr, O-sulpho-Tyr, O-phospho-Tyr or nitro-Tyr, Z1 is des-Z1 or a peptide X1-Y1, Z2 is des-Z2 or a peptide X2-Y2, Z3 is des-Z3 or a peptide X3-Y1, Z4 is des-Z4 or a peptide X4-Y1, Z5 is des-Z5 or a peptide X5-Y3, Z6 is des-Z6 or a peptide X6-Y1, Z7 is des-Z7 or a peptide X7-Y1, Z8 is des-Z8 or a peptide X8-Y1, Z9 is des-Z9 or a peptide X2-Y1, Z10 is des-Z10 or a peptide X9-Y1, Z11 is des-Z11 or a peptide X10-Y1, Z12 is des-Z12 or a peptide X10-Y4, Z13 is des-Z13 or a peptide X11-Y1, Z14 is des-Z14 or a peptide X12-Y1, Z15 is des-Z15 or a peptide X12-Y1, X1 is Asn-Lys-Lys-Lys-Xaa3 (SEQ ID NO:26), X2 is Arg-Xaa3-Lys-Lys-Lys-Lys-Xaa3 (SEQ ID NO:27), X3 is Xaa3-Xaa3-Lys-Lys-Lys-Lys-Arg-Xaa3 (SEQ ID NO:28), X4 is Xaa3-His-Gln-Lys-Lys-Lys-Arg-Xaa3 (SEQ ID NO:29), X5 is Lys-Xaa3-Lys-Lys-Xaa3-Lys-Xaa3 (SEQ ID NO:30), X6 is Xaa3-Xaa3-Lys-Lys-Lys-Lys-Xaa3 (SEQ ID NO:31), X7 is Ser-His-Gln-Arg-Lys-Lys-Xaa3 (SEQ ID NO:32), X8 is Xaa3-Xaa3-Lys-Arg-Lys-Xaa3 (SEQ ID NO:33), X9 is Lys-Xaa3-Thr-Lys-Lys-Arg-Xaa3 (SEQ ID NO:34), X10 is Lys-Xaa3-Lys-Xaa3-Lys-Lys-Ser (SEQ ID NO:35), X11 is Xaa3-Thr-Lys-Xaa3-Lys-Lys-Xaa3 (SEQ ID NO:36), X12 is Ser-Xaa3-Lys-Lys-Lys-Lys-Xaa3 (SEQ ID NO:37), X13 is Xaa3-His-Gln-Arg-Lys-Lys-Xaa3 (SEQ ID NO:38), Y1 is Gly-Arg-Arg-Asn-Asp (SEQ ID NO:39), Y2 is Gly-His-Arg-Asn-Asp (SEQ ID NO:40), Y3 is Gly-Lys or Gly-Lys-Gly-Arg-Arg-Asn-Asp (SEQ ID NO:41), and Y4 is Gly-Arg-Arg-Asn-His (SEQ ID NO:42).

9. The method of claim 8, wherein said κA conopeptide is further modified to comprise an O-glycan.

10. An isolated κA conopeptide seleceted from the group consisting of:
(SEQ ID NO:1)-Z1
κA A10.1: Xaa2-Lys-Xaa1-Leu-Val-Val-Thr-Ala-Thr-
Thr-Thr-Cys-Cys-Gly-Xaa5-Asn-Xaa3-Met-Thr-Ser-
Cys-Xaa3-Arg-Cys-Met-Cys-Asp-Ser-Cys;
(SEQ ID NO:2)-Z2
κA A10.2: Xaa2-Xaa3-Xaa4-Leu-Val-Xaa3-Ser-Lys-
Ile-Thr-Asn-Cys-Cys-Gly-Xaa5-Asn-Asn-Met-Xaa1-Met-
Cys-Xaa3-Thr-Cys-Met-Cys-Thr-Xaa5-Ser-Cys;
(SEQ ID NO:3)-Z3
κA C10.1a: Xaa2-Lys-Xaa1-Leu-Val-Xaa3-Ser-Thr-
Ile-Thr-Thr-Cys-Cys-Gly-Asn-Xaa1-Xaa3-Gly-Thr-Met-
Cys-Xaa3-Lys-Cys-Met-Cys-Asp-Asn-Thr-Cys;
(SEQ ID NO:4)-Z3
κA C10.1b: Xaa2-Lys-Xaa1-Leu-Val-Xaa3-Ser-Thr-Ile-
Thr-Thr-Cys-Cys-Gly-His-Xaa1-Xaa3-Gly-Thr-Met-Cys-
Xaa3-Lys-Cys-Met-Cys-Asp-Asn-Thr-Cys;
(SEQ ID NO:5)-Z4
κA C10.2: Xaa2-Lys-Xaa1-Leu-Val-Val-Thr-Ala-Thr-
Thr-Thr-Cys-Cys-Gly-Xaa5-Asn-Xaa3-Met-Ser-Met-Cys-
Xaa3-Lys-Cys-Met-Cys-Thr-Xaa5-Ser-Cys;
(SEQ ID NO:6)-Z5
κA Cr10.1: Xaa2-Xaa3-Xaa4-Leu-Val-Xaa3-Ser-Thr-
Ile-Thr-Thr-Cys-Cys-Gly-Xaa5-Asp-Xaa3-Gly-Thr-Lys-
Cys-Xaa3-Xaa3-Cys-Arg-Cys-Asn-Asn-Thr-Cys;
(SEQ ID NO:7)
κA Cn10.1: Ala-Xaa3-Xaa4-Leu-Val-Xaa3-Ser-Gln-Ile-
Thr-Thr-Cys-Cys-Gly-Xaa5-Asn-Xaa3-Gly-Thr-Met-Cys-
Xaa3-Ser-Cys-Met-Cys-Thr-Asn-Ser-Cys;
(SEQ ID NO:8)-Z6
κA Cn10.2: Xaa2-Lys-Asp-Leu-Val-Val-Thr-Ala-Thr-
Thr-Thr-Cys-Cys-Gly-Xaa5-Asn-Xaa3-Met-Thr-Ile-Cys-
Xaa3-Xaa3-Cys-Met-Cys-Thr-Xaa5-Ser-Cys;
(SEQ ID NO:9)-Z7
κA M10.2: Ala-Xaa3-Xaa1-Leu-Val-Val-Thr-Ala-Thr-
Thr-Thr-Cys-Cys-Gly-Phe-Asp-Xaa3-Met-Thr-Xaa4-Cys-
Xaa3-Xaa3-Cys-Met-Cys-Thr-Xaa5-Ser-Cys;
(SEQ ID NO:10)-Z8
κA U006: Ala-Xaa3-Xaa1-Leu-Val-Val-Thr-Ala-Thr-
Thr-Asn-Cys-Cys-Gly-Xaa5-Asn-Xaa3-Met-Thr-Ile-Cys-
Xaa3-Xaa3-Cys-Met-Cys-Thr-Xaa5-Ser-Cys;
(SEQ ID NO:11)-Z1
κA Mn10.1: Xaa2-Lys-Xaa1-Leu-Val-Val-Thr-Ala-Thr-
Thr-Thr-Cys-Cys-Gly-Xaa5-Asn-Xaa3-Met-Thr-Ser-Cys-
Xaa3-Arg-Cys-Met-Cys-Asp-Ser-Ser-Cys;
(SEQ ID NO:12)-Z9
κA Mn10.2: Xaa2-Xaa3-Xaa4-Leu-Val-Xaa3-Ser-Lys-
Ile-Thr-Asn-Cys-Cys-Gly-Xaa5-Asn-Thr-Met-Xaa1-Met-
Cys-Xaa3-Thr-Cys-Met-Cys-Thr-Xaa5-Ser-Cys;
(SEQ ID NO:13)-Z10
κA Sm10.2: Ala-Xaa3-Xaa4-Leu-Val-Xaa3-Ser-Thr-Ile-
Thr-Thr-Cys-Cys-Gly-Xaa5-Asp-Xaa3-Gly-Thr-Met-Cys-
Xaa3-Xaa3-Cys-Met-Cys-Asn-Asn-Thr-Cys;
(SEQ ID NO:14)-Z11
κA Sm10.3: Xaa2-Ala-Xaa3-Leu-Val-Xaa3-Ser-Thr-Ile-
Thr-Thr-Cys-Cys-Gly-Xaa5-Asp-Xaa3-Gly-Thr-Met-Cys-Xaa3-Thr-
Cys-Met-Cys-Asn-Asn-Cys;
(SEQ ID NO:15)-Z11
κA SmVIII: Xaa2-Thr-Xaa4-Leu-Val-Xaa3-Ser-Thr-Ile-
Thr-Thr-Cys-Cys-Gly-Xaa5-Asp-Xaa3-Gly-Thr-Met-Cys-
Xaa3-Thr-Cys-Met-Cys-Asp-Asn-Cys;
(SEQ ID NO:16)-Z12
κA SmVIIIA: Ala-Xaa3-Xaa4-Leu-Val-Xaa3-Ser-Thr-
Ile-Thr-Cys-Cys-Gly-Xaa5-Asp-Xaa3-Gly-Ser-Met-
Cys-Xaa3-Xaa3-Cys-Met-Cys-Asn-Asn-Thr-Cys;
(SEQ ID NO:17)
κA SIVA: Xaa2-Lys-Ser-Leu-Val-Xaa3-Ser-Val-Ile-
Thr-Thr-Cys-Cys-Gly-Xaa5-Asp-Xaa3-Gly-Thr-Met-Cys-
Xaa3-Xaa3-Cys-Arg-Cys-Thr-Asn-Ser-Cys;
(SEQ ID NO:18)-Z13
κA SVIIIA: Xaa2-Lys-Xaa1-Leu-Val-Xaa3-Ser-Val-Ile-
Thr-Thr-Cys-Cys-Gly-Xaa5-Asp-Xaa3-Gly-Thr-Met-Cys-
Xaa3-Xaa3-Cys-Arg-Cys-Thr-Asn-Ser-Cys;
(SEQ ID NO:19)
κA Sx10.1: Xaa2-Ser-Xaa4-Leu-Val-Xaa3-Ser-Thr-Ile-
Thr-Thr-Cys-Cys-Gly-Xaa5-Ser-Xaa3-Gly-Thr-Met-Cys-
Xaa3-Xaa3-Cys-Met-CysThr-Asn-Thr-Met;
(SEQ ID NO:20)-Z14
κA S110.1: Xaa2-Lys-Asp-Leu-Val-Val-Thr-Ala-Thr-
Thr-Cys-Cys0Gly-Xaa5-Asn-Xaa3-Met-Thr-Met-Cys-
Xaa3-Xaa3-Cys-Met-Arg-Thr-Xaa5-Ser-Cys;
(SEQ ID NO:21)-Z13
κA S110.2: Xaa2-Lys-Xaa1-Leu-Val-Xaa3-Ser-Val-Ile-
Thr-Thr-Cys-Cys-Gly-Xaa5-Asp-Xaa3-Gly-Thr-Met-Cys-
Xaa3-Xaa3-Cys-Arg-Cys-Asn-Ser-Cys;
(SEQ ID NO:22)
κA A671: Ala-Xaa3-Xaa4-Leu-Val-Xaa3-Ser-Thr-Ile-
Thr-Thr-Cys-Cys-Gly-Xaa5-Asn-Xaa3-Gly-Thr-Met-Cys-
Xaa3-Xaa3-Cys-Arg-Cys-Asp-Asn-Thr-Cys;
(SEQ ID NO:23)-Z5
κA H350: Xaa2-Ser-Xaa4-Leu-Val-Xaa3-Ser-Thr-Ile-
Thr-Thr-Cys-Cys-Gly-Xaa5-Asp-Xaa3-Gly-Thr-Met-Cys-
Xaa3-Xaa3-Cys-Arg-Cys-Asn-Asn-Thr-Cys;
(SEQ ID NO:24)-Z8
κA J454: Ala-Xaa3-Xaa1-Leu-Val-Val-Thr-Ala-Thr-
Thr-Thr-Cys-Cys-Gly-Xaa5-Asp-Xaa3-Met-Thr-Ile-Cys-
Xaa3-Xaa3-Cys-Met-Cys-Thr-His-Ser-Cys;
and
(SEQ ID NO:25)-Z15
κA G851: Ala-Xaa3-Xaa1-Leu-Val-Val-Thr-Ala-Thr-
Thr-Thr-Cys-Cys-Gly-Xaa5-Asp-Xaa3-Met-Thr-Xaa4-
Cys-Xaa3-Ser-Cys-Met-Cys-Thr-Xaa5-Ser-Cys,
wherein Xaa1 is Glu or γ-carboxy-Glu, Xaa2 is Gln or pyro-Glu, Xaa3 is Pro or hydroxy-Pro, Xaa4 is Trp, D-Trp orbromo-Trp, Xaa5 is Tyr, mono-iodo-Tyr, di-iodo-tyr, O-sulpho-Tyr, O-phospho-Tyr or nitro-Tyr, Z1 is des-Z1 or a peptide X1-Y1, Z2 is des-Z2 or a peptide X2-Y2, Z3 is des-Z3 or a peptide X3-Y1, Z4 is des-Z4 or a peptide X4-Y1, Z5 is des-Z5 or a peptide X5-Y3, Z6 is des-Z6 or a peptide X6-Y1, Z7 is des-Z7 or a peptide X7-Y1, Z8 is des-Z8 or a peptide X8-Y1, Z9 is des-Z9 or a peptide X2-Y1, Z10 is des-Z10 or a peptide X9-Y1, Z11 is des-Z11 or a peptide X10-Y1, Z12 is des-Z12 or a peptide X10-Y4, Z13 is des-Z13 or a peptide X11-Y1, Z14 is des-Z14 or a peptide X12-Y1, Z15 is des-Z15 or a peptide X12-Y1, X1 is Asn-Lys-Lys-Lys-Xaa3 (SEQ ID NO:26), X2 is Arg-Xaa3-Lys-Lys-Lys-Lys-Xaa3 (SEQ ID NO:27), X3 is Xaa3-Xaa3-Lys-Lys-Lys-Lys-Arg-Xaa3 (SEQ ID NO:28), X4 is Xaa3-His-Gln-Lys-Lys-Lys-Arg-Xaa3 (SEQ ID NO:29), X5 is Lys-Xaa3-Lys-Lys-Xaa3-Lys-Xaa3 (SEQ ID NO:30), X6 is Xaa3-Xaa3-Lys-Lys-Lys-Lys-Xaa3 (SEQ ID NO:31), X7 is Ser-His-Gln-Arg-Lys-Lys-Xaa3 (SEQ ID NO:32), X8 is Xaa3-Xaa3-Lys-Arg-Lys-Xaa3 (SEQ ID NO:33), X9 is Lys-Xaa3-Thr-Lys-Lys-Arg-Xaa3 (SEQ ID NO:34), X10 is Lys-Xaa3-Lys-Xaa3-Lys-Lys-Ser (SEQ ID NO:35), X11 is Xaa3-Thr-Lys-Xaa3-Lys-Lys-Xaa3 (SEQ ID NO:36), X12 is Ser-Xaa3-Lys-Lys-Lys-Lys-Xaa3 (SEQ ID NO:37), X13 is Xaa3-His-Gln-Arg-Lys-Lys-Xaa3 (SEQ ID NO:38), Y1 is Gly-Arg-Arg-Asn-Asp (SEQ ID NO:39), Y2 is Gly-His-Arg-Asn-Asp (SEQ ID NO:40), Y3 is Gly-Lys or Gly-Lys-Gly-Arg-Arg-Asn-Asp (SEQ ID NO:41), and Y4 is Gly-Arg-Arg-Asn-His (SEQ ID NO:42).

11. The isolated κA conopeptide of claim 10, wherein said κA conopeptide is further modified to comprise an O-glycan.

12. An isolated nucleic acid comprising a nucleic acid coding for an κA conopeptide precursor comprising an amino acid sequence selected from the group of amino acid sequences set forth in Tables 2-25.

13. The nucleic acid of claim 12 wherein the nucleic acid comprises a nucleotide sequence selected from the group of nucleotide sequences set forth in Tables 2-25 or their complements.

14. An isolated κA conopeptide precursor comprising an amino acid sequence selected from the group of amino acid sequences set forth in Tables 2-14, 16-18, 21, and 23-25.

15. The isolated κA conopeptide precursor of claim 14, wherein the precursor comprises the amino acid sequence set forth in SEQ ID NO:51.

16. The isolated κA conopeptide of claim 10, wherein the κA conopeptide is
(SEQ ID NO:22)
κA A671: Ala-Xaa3-Xaa4-Leu-Val-Xaa3-Ser-Thr-
Ile-Thr-Thr-Cys-Cys-Gly-Xaa5-Asn-Xaa3-Gly-Thr-Met-
Cys-Xaa3-Xaa3-Cys-Arg-Cys-Asp-Asn-Thr-Cys.

17. The method of claim 8, wherein the κA conopeptide is
(SEQ ID NO :22)
κA A671: Ala-Xaa3-Xaa4-Leu-Val-Xaa3 -Ser-Thr-
Ile-Thr-Thr-Cys-Cys-Gly-Xaa5-Asn-Xaa3-Gly-Thr-Met-
Cys-Xaa3-Xaa3-Cys-Arg-Cys-Asp-Asn-Thr-Cys.


SEQUENCE LISTING (97 sequences)

SEQ ID NO: 1 - protein, 30 aa, Conus achatinus
  PEPTIDE (1)..(15): Xaa at residue 1 is Gln or pyro-Glu; Xaa at residue 3 is Glu or gamma-carboxy-Glu; Xaa at residue 15 is Tyr, mono-iodo-Tyr, di-iodo-Tyr, O-sulpho-Tyr, O-phospho-Tyr or nitro-Tyr.
XKXLVVTATTTCCGXNXMTSCXRCMCDSSC

SEQ ID NO: 2 - protein, 30 aa, Conus achatinus
  PEPTIDE (1)..(21): Xaa at residue 1 is Gln or pyro-Glu; Xaa at residues 2, 6 and 22 is Pro or hydroxy-Pro; Xaa at residue 3 is Trp, D-Trp or bromo-Trp.
XXXLVXSKITNCCGXNNMXMCXTCMCTXSC

SEQ ID NO: 3 - protein, 30 aa, Conus catus
  PEPTIDE (1)..(30): Xaa at residue 1 is Gln or pyro-Glu; Xaa at residues 3 and 16 is Glu or gamma-carboxy-Glu; Xaa at residues 6, 17 and 22 is Pro or hydroxy-Pro.
XKXLVXSTITTCCGNXXGTMCXKCMCDNTC

SEQ ID NO: 4 - protein, 30 aa, Conus catus
  PEPTIDE (1)..(30): Xaa at residue 1 is Gln or pyro-Glu; Xaa at residues 3 and 16 is Glu or gamma-carboxy-Glu; Xaa at residues 6, 17 and 22 is Pro or hydroxy-Pro.
XKXLVXSTITTCCGHXXGTMCXKCMCDNTC

SEQ ID NO: 5 - protein, 30 aa, Conus catus
  PEPTIDE (1)..(28): Xaa at residue 1 is Gln or pyro-Glu; Xaa at residue 3 is Glu or gamma-carboxy-Glu; Xaa at residues 15 and 28 is Tyr, mono-iodo-Tyr, di-iodo-Tyr, O-sulpho-Tyr, O-phospho-Tyr or nitro-Tyr.
XKXLVVTATTTCCGXNXMSMCXKCMCTXSC

SEQ ID NO: 6 - protein, 30 aa, Conus circumcisus
  PEPTIDE (1)..(22): Xaa at residue 1 is Gln or pyro-Glu; Xaa at residues 2, 6, 17,  22 and 23 is Pro or hydroxy-Pro; Xaa at residue 3 is Trp, D-Trp or bromo-Trp.
XXXLVXSTITTCCGXDXGTKCXXCRCNNTC

SEQ ID NO: 7 - protein, 30 aa, Conus consors
  PEPTIDE (2)..(22): Xaa at residues 2, 6, 17 and 22 is Pro or hydroxy-Pro; Xaa at residue 3 is Trp, D-Trp or breomo-Trp; Xaa at residue 15 is Tyr, mono-iodo-Tyr, di-iodo-Tyr, O-sulpho-Tyr, O-phospho-Tyr or nitro-Tyr.
AXXLVXSQITTCCGXNXGTMCXSCMCTNSC

SEQ ID NO: 8 - protein, 30 aa, Conus consors
  PEPTIDE (1)..(28): Xaa at residue 1 is Gln or pyro-Glu; Xaa at residues 15 and 28 is Tyr, mono-iodo-Tyr, di-iodo-Tyr, O-sulpho-Tyr, O-phospho-Tyr or nitro-Tyr; Xaa at residues 17, 22 and 23 is Pro or hydroxy-Pro.
XKDLVVTATTTCCGXNXMTICXXCMCTXSC

SEQ ID NO: 9 - protein, 30 aa, Conus magus
  PEPTIDE (2)..(23): Xaa at residues 2, 17, 22 and 23 is Pro or hydroxy-Pro; Xaa at residue 3 is Glu or gamma-carboxy-Glu; Xaa at residue 20 is Trp, D-Trp or bromo-Trp.
AXXLVVTATTTCCGFDXMTXCXXCMCTXSC

SEQ ID NO: 10 - protein, 30 aa, Conus magus
  PEPTIDE (2)..(23): Xaa at residues 2, 17, 22 and 23 is Pro or hydroxy-Pro; Xaa at residue 3 is Glu or gamma-carboxy-Glu.
AXXLVVTATTNCCGXNXMTICXXCMCTXSC

SEQ ID NO: 11 - protein, 30 aa, Conus monachus; identical to SEQ ID NO: 1
  PEPTIDE (1)..(15): Xaa at residue 1 is Gln or pyro-Glu; Xaa at residue 3 is Glu or gamma-carboxy-Glu; Xaa at residue 15 is Tyr, mono-iodo-Tyr, di-iodo-Tyr, O-sulpho-Tyr, O-phospho-Tyr or nitro-Tyr.

SEQ ID NO: 12 - protein, 30 aa, Conus monachus
  PEPTIDE (1)..(22): Xaa at residue 1 is Gln or pyro-Glu; Xaa at residue 2, 6 and 22 is Pro or hydroxy-Pro; Xaa at residue 3 is Trp, D-Trp or bromo-Trp.
XXXLVXSKITNCCGXNTMXMCXTCMCTXSC

SEQ ID NO: 13 - protein, 30 aa, Conus stercusmuscarum
  PEPTIDE (2)..(30): Xaa at residues 2, 6, 17, 22 and 23 is Pro or hydroxy-Pro; Xaa at residue 15 is Tyr, mono-iodo-Tyr, di-iodo- Tyr, O-sulpho-Tyr, O-phospho-Tyr or nitro-Tyr; Xaa at residue 3 is Trp, D-Trp or bromo-Trp.
AXXLVXSTITTCCGXDXGTMCXXCMCNNTC

SEQ ID NO: 14 - protein, 30 aa, Conus stercusmuscarum
  PEPTIDE (1)..(22): Xaa at residue 1 is Gln or pyro-Gln; Xaa at residues 3, 6, 17 and 22 is Pro or hydroxy-Pro; Xaa at residue 15 is Tyr, mono-iodo-Tyr, di-iodo-Tyr, O-sulpho-Tyr, O-phospho-Tyr or nitro-Tyr.
XAXLVXSTITTCCGXDXGTMCXTCMCNNTC

SEQ ID NO: 15 - protein, 30 aa, Conus stercusmuscarum
  PEPTIDE (1)..(22): Xaa at residue 1 is Gln or pyro-Glu; Xaa at residue 3 is Trp, D-Trp or bromo-Trp; Xaa at residues 6, 17 and 22 is Pro or hydroxy-Pro.
XTXLVXSTITTCCGXDXGTMCXTCMCDNTC

SEQ ID NO: 16 - protein, 30 aa, Conus stercusmuscarum
  PEPTIDE (2)..(23): Xaa at residues 2, 6, 17, 22 and 23 is Pro or hydroxy-Pro; Xaa at residue 3 is Trp, D-Trp or bromo-Trp; Xaa at residue 15 is Tyr, mono-iodo-Tyr, di-iodo-Tyr, O-sulpho-Tyr, O-phospho-Tyr or nitro-Tyr.
AXXLVXSTITTCCGXDXGSMCXXCMCNNTC

SEQ ID NO: 17 - protein, 30 aa, Conus striatus
  PEPTIDE (1)..(23): Xaa at residue 1 is Gln or pyro-Glu; Xaa at residues 6, 17, 22 and 23 is Pro or hydroxy-Pro; Xaa at residue 15 is Tyr, mono-iodo-Tyr, di-iodo-Tyr, O-sulpho-Tyr, O-phospho-Tyr or nitro-Tyr.
XKSLVXSVITTCCGXDXGTMCXXCRCTNSC

SEQ ID NO: 18 - protein, 30 aa, Conus striatus
  PEPTIDE (1)..(23): Xaa at residue 1 is Gln or pyro-Glu; Xaa at residue 3 is Glu or gamma-carboxy-Glu; Xaa at residues 6, 17, 22 and 23 is Pro or hydroxy-Pro.
XKXLVXSVITTCCGXDXGTMCXXCRCTNSC

SEQ ID NO: 19 - protein, 30 aa, Conus striolatus
  PEPTIDE (1)..(23): Xaa at residue 1 is Gln or pyro-Glu; Xaa at residue 3 is Trp, D-Trp or bromo-Trp; Xaa at residues 6, 17, 22 and 23 is Pro or hydroxy-Pro.
XSXLVXSTITTCCGXSXGTMCXXCMCTNTC

SEQ ID NO: 20 - protein, 30 aa, Conus sulcatus
  PEPTIDE (1)..(28): Xaa at residue 1 is Gln or pyro-Glu; Xaa at residues 15 and 28 is Tyr, mono-iodo-Tyr, di-iodo-Tyr, O-sulpho-Tyr, O-phospho-Tyr or nitro-Tyr.
XKDLVVTATTTCCGXNXMTMCXXCMRTXSC

SEQ ID NO: 21 - protein, 30 aa, Conus sulcatus; identical to SEQ ID NO: 18
  PEPTIDE (1)..(23): Xaa at residue 1 is Gln or pyro-Glu; Xaa at residue 3 is Glu or gamma-carboxy-Glu; Xaa at residues 6, 17, 22 and 23 is Pro or hydroxy-Pro.

SEQ ID NO: 22 - protein, 30 aa, Conus aurisiacus
  PEPTIDE (2)..(23): Xaa at residues 2, 6, 17, 22 and 23 is Pro or hydroxy-Pro; Xaa at residue 3 is Trp, D-Trp or bromo-Trp; Xaa at residue 15 is Try, mono-iodo-Tyr, di-iodo-Tyr, O-sulpho-Tyr, O-phospho-Tyr or nitro-Tyr.
AXXLVXSTITTCCGXNXGTMCXXCRCDNTC

SEQ ID NO: 23 - protein, 30 aa, Conus aurisiacus
  PEPTIDE (1)..(23): Xaa at residue 1 is Gln or pyro-Glu; Xaa at residue 3 is Trp, D-Trp or bromo-Trp; Xaa at residues 6, 17, 22 and 23 is Pro or hydroxy-Pro.
XSXLVXSTITTCCGXDXGTMCXXCRCNNTC

SEQ ID NO: 24 - protein, 30 aa, Conus consors
  PEPTIDE (2)..(23): Xaa at residues 2, 17, 22 and 23 is Pro or hydroxy-Pro; Xaa at residue 3 is Glu or gamma-carboxy-Glu; Xaa at residue 15 is Tyr, mono-iodo-Tyr, di-iodo-Tyr, O-sulpho-Tyr, O-phospho-Tyr or nitro-Tyr.
AXXLVVTATTTCCGXDXMTICXXCMCTHSC

SEQ ID NO: 25 - protein, 30 aa, Conus consors
  PEPTIDE (2)..(22): Xaa at residues 2, 17 and 22 is Pro or hydroxy-Pro; Xaa at residue 3 is Glu or gamma-carboxy-Glu; Xaa at residue 20 is Trp, D-Trp or bromo-Trp.
AXXLVVTATTTCCGXDXMTXCXSCMCTXSC

SEQ ID NO: 26 - protein, 5 aa, Artificial Sequence
  Description of Artificial SequenceConus internal peptide
NKKKX

SEQ ID NO: 27 - protein, 7 aa, Artificial Sequence
  Description of Artificial SequenceConus internal peptide
RXKKKKX

SEQ ID NO: 28 - protein, 8 aa, Artificial Sequence
  Description of Artificial SequenceConus internal peptide
XXKKKKRX

SEQ ID NO: 29 - protein, 8 aa, Artificial Sequence
  Description of Artificial SequenceConus internal peptide
XHQKKKRX

SEQ ID NO: 30 - protein, 7 aa, Artificial Sequence
  Description of Artificial SequenceConus internal peptide
KXKKXKX

SEQ ID NO: 31 - protein, 7 aa, Artificial Sequence
  Description of Artificial SequenceConus internal peptide
XXKKKKX

SEQ ID NO: 32 - protein, 7 aa, Artificial Sequence
  Description of Artificial SequenceConus internal peptide
SHQRKKX

SEQ ID NO: 33 - protein, 6 aa, Artificial Sequence
  Description of Artificial SequenceConus internal peptide
XXKRKX

SEQ ID NO: 34 - protein, 7 aa, Artificial Sequence
  Description of Artificial SequenceConus internal peptide
KXTKKRX

SEQ ID NO: 35 - protein, 7 aa, Artificial Sequence
  Description of Artificial SequenceConus internal peptide
KXKXKKS

SEQ ID NO: 36 - protein, 7 aa, Artificial Sequence
  Description of Artificial SequenceConus internal peptide
XTKXKKX

SEQ ID NO: 37 - protein, 7 aa, Artificial Sequence
  Description of Artificial SequenceConus internal peptide
SXKKKKX

SEQ ID NO: 38 - protein, 7 aa, Artificial Sequence
  Description of Artificial SequenceConus internal peptide
XHQRKKX

SEQ ID NO: 39 - protein, 5 aa, Artificial Sequence
  Description of Artificial SequenceConus C-terminus
GRRND

SEQ ID NO: 40 - protein, 5 aa, Artificial Sequence
  Description of Artificial SequenceConus C-terminus
GHRND

SEQ ID NO: 41 - protein, 7 aa, Artificial Sequence
  Description of Artificial SequenceConus C-terminus
GKGRRND

SEQ ID NO: 42 - protein, 5 aa, Artificial Sequence
  Description of Artificial SequenceConus C-terminus
GRRNH

SEQ ID NO: 43 - protein, 30 aa, Artificial Sequence
  Description of Artificial SequenceGeneric Conus KappaA conopeptide
XXXLVXXXXTTCCGXXXXXXCXXCXCXXXC

SEQ ID NO: 44 - protein, 30 aa, Artificial Sequence
  Description of Artificial SequenceConus Kappa conopeptide consensus
XXXLVXSXITTCCGYDXGTMCXXCXCTNXC

SEQ ID NO: 45 - protein, 27 aa, Conus striatus
  PEPTIDE (6)..(25): Xaa at residues 6, 11, 12, 20, 23 and 25 is unknown; Xaa at residues 16, 21 and 22 is hydroxy-Pro.
KSLVPXVITTXXGYDXGTMXXXXRXTN

SEQ ID NO: 46 - protein, 25 aa, Conus striatus
  PEPTIDE (6)..(22): Xaa at residue 6 is unknown; Xaa at residues 16, 21 and 22 is hydroxy-Pro.
KSLVPXVITTCCGYDXGTMCXXCRC

SEQ ID NO: 47 - protein, 30 aa, Conus striatus
  PEPTIDE (1)..(23): Xaa at residue 1 is pyro-Glu; Xaa at residues 17, 22 and 23 is hydroxy-Pro.
XKSLVPSVITTCCGYDXGTMCXXCRCTNSC

SEQ ID NO: 48 - DNA, 30 bp, Artificial Sequence
  Description of Artificial SequenceConus A family primer
caggatccatgttcaccgtgtttctgttgg

SEQ ID NO: 49 - DNA, 25 bp, Artificial Sequence
  Description of Artificial SequenceConus A family primer
atctcgagcatcagtcgtttctgcg

SEQ ID NO: 50 - DNA, 434 bp, Conus aurisiacus
  CDS (1)..(189)
atgttcaccgtgtttctgttggttgtcttggcaaccactgtcgtttccatcccttcagatcgtgcatctgatggcaggaatgccgcagtcaacgagagagcgccttggctggtcccttcgacaatcacgacttgctgtggatataatccggggacaatgtgccctccttgcaggtgcgataatacctgttaaccaaaaaaaaccaaaaccaggccgcagaaacgactgatgctccaggaccctctgaaccacgacatgccgccctctgcctgacctgcttcactttccgtctctttgtgccactagaactgtacaactcgatccactagactcccacgttacctccgtattctgaaactacttggatttgattgtccttaatatctgctcatacttgctgttattacatcgtccaaaaaaaaaaaaaaaaaaaa

SEQ ID NO: 51 - protein, 63 aa, Conus aurisiacus
MFTVFLLVVLATTVVSIPSDRASDGRNAAVNERAPWLVPSTITTCCGYNPGTMCPPCRCDNTC

SEQ ID NO: 52 - DNA, 272 bp, Conus aurisiacus
  CDS (7)..(237)
ggatccatgttcaccgtgtttctgttggttgtcttggcaaccactgtcgtttccatcccttcagatcgtgcatctgatggcaggaatgccgcagtcaacgagagacaatcttggctggtcccttcgacaatcacgacttgctgtggatatgatccggggacaatgtgccctccttgcaggtgcaataatacctgtaaaccaaaaaaaccaaaaccaggaaaaggccgcagaaacgactgatgctccaggaccctctgaaccacgacctcgag

SEQ ID NO: 53 - protein, 77 aa, Conus aurisiacus
MFTVFLLVVLATTVVSIPSDRASDGRNAAVNERQSWLVPSTITTCCGYDPGTMCPPCRCNNTCKPKKPKPGKGRRND

SEQ ID NO: 54 - DNA, 239 bp, Conus consors
  CDS (7)..(228)
ggatccatgttcaccgtgtttctgttggttgtcttggcaaccactgtcgtttccatcccttcagatcgtgcatctgaaggcaggaatgccgtagtccacgagagagcgcctgagctggtcgttacggccaccacgacttgctgtggttatgatccgatgacaatatgccctccttgcatgtgcactcattcctgtccaccaaaaagaaaaccaggccgcagaaacgactgatgctcgag

SEQ ID NO: 55 - protein, 74 aa, Conus consors
MFTVFLLVVLATTVVSIPSDRASEGRNAVVHERAPELVVTATTTCCGYDPMTICPPCMCTHSCPPKRKPGRRND

SEQ ID NO: 56 - DNA, 266 bp, Conus consors
  CDS (7)..(231)
ggatccatgttcaccgtgtttctgttggttgtcttggcaaccactgtcgtttccatcccttcagatcgtgcatctgatggcaggaatgccgtagtccacgagagagcgcctgagctggtcgttacggccaccacgacttgctgtggttatgatccgatgacatggtgcccttcttgcatgtgcacttattcctgtccccaccaaaggaaaaaaccaggccgcagaaacgactgatgctccaggaccctctgaaccacgacctcgag

SEQ ID NO: 57 - protein, 75 aa, Conus consors
MFTVFLLVVLATTVVSIPSDRASDGRNAVVHERAPELVVTATTTCCGYDPMTWCPSCMCTYSCPHQRKKPGRRND

SEQ ID NO: 58 - DNA, 250 bp, Conus achatinus
  CDS (1)..(219)
atgttcaccgtgtttctgttggttgtcttggcaaccactctcgtttccatcccttcagatcgtgcatctgatttcaggaatgccgcagtccacgagagacagaaggagctggtcgttacggccaccacgacttgctgtggttataatccgatgacatcgtgccctcgttgcatgtgcgatagtagctgcaacaagaaaaaaccaggccgcagaaacgactgatgctccaggaccctctgaaccacgacgt

SEQ ID NO: 59 - protein, 73 aa, Conus achatinus
MFTVFLLVVLATTLVSIPSDRASDFRNAAVHERQKELVVTATTTCCGYNPMTSCPRCMCDSSCNKKKPGRRND

SEQ ID NO: 60 - DNA, 256 bp, Conus achatinus
  CDS (1)..(225)
atgttcaccgtgtttctgttggttgtcttggcaaccactctcgtttccatcccttcagatcgtgcatctgatggcaggaatgccgtagtccacgagagacagccttggctggtcccttcgaaaatcacgaattgctgtggttataataacatggaaatgtgccctacttgcatgtgcacttattcctgtcgccccaaaaagaaaaaaccaggccacagaaacgactgatgctccaggaccctctgaaccacgacgt

SEQ ID NO: 61 - protein, 75 aa, Conus achatinus
MFTVFLLVVLATTLVSIPSDRASDGRNAVVHERQPWLVPSKITNCCGYNNMEMCPTCMCTYSCRPKKKKPGHRND

SEQ ID NO: 62 - DNA, 259 bp, Conus catus
  CDS (1)..(228)
atgttcaccgtgtttctgttggttggcttggcaaccactctcgtttccattccttcagatggtgcatctgatggcaagaatgccgcagtccacgagagacagaaggagctggtcccttcgacaatcacgacttgctgtggtaatgaaccggggacaatgtgccctaaatgcatgtgcgataatacctgtccccccaaaaagaagaaaagaccaggccgcagaaacgactgatgctccaggaccctctgaaccacgacgt

SEQ ID NO: 63 - protein, 76 aa, Conus catus
MFTVFLLVGLATTLVSIPSDGASDGKNAAVHERQKELVPSTITTCCGNEPGTMCPKCMCDNTCPPKKKKRPGRRND

SEQ ID NO: 64 - DNA, 259 bp, Conus catus
  CDS (1)..(228)
atgttcaccgtgtttctgttggttggcttggcaaccactctcgtttccattccttcagatggtgcatctgatggcaagaatgccgcagtccacgagagacagaaggagctggtcccttcgacaatcacgacttgctgtggtcatgaaccggggacaatgtgccctaaatgcatgtgcgataatacctgtccccccaaaaagaagaaaagaccaggccgcagaaacgactgatgctccaggaccctctgaaccacgacgt

SEQ ID NO: 65 - protein, 76 aa, Conus catus
MFTVFLLVGLATTLVSIPSDGASDGKNAAVHERQKELVPSTITTCCGHEPGTMCPKCMCDNTCPPKKKKRPGRRND

SEQ ID NO: 66 - DNA, 259 bp, Conus catus
  CDS (1)..(228)
atgttcaccgtgtttctgttggttggcttggcaaccactctcgtttccattccttcagatggtgcatctgatgtcaggaatgccgcagtcctcgagagacagaaggagctggtcgttacggccaccacgacttgctgtggttataatccgatgtcaatgtgccctaaatgcatgtgcacttattcctgtccccaccaaaagaagaaaagaccaggccgcagaaacgactgatgctccaggaccctctgaaccacgacgt

SEQ ID NO: 67 - protein, 76 aa, Conus catus
MFTVFLLVGLATTLVSIPSDGASDVRNAAVLERQKELVVTATTTCCGYNPMSMCPKCMCTYSCPHQKKKRPGRRND

SEQ ID NO: 68 - DNA, 261 bp, Conus circumcisus
  CDS (1)..(231)
atgttcaccgtgtttctgttggttgtcttggcaaccactgtcgtttccatcccttcagatcgtgcatctgatggcaggaatgccgcagtcaacgagagacaaccttggctggtcccttcgacaatcacgacttgctgtggatatgatccggggacaaagtgccctccttgcaggtgcaataatacctgtaaaccaaaaaaaccaaaaccaggaaaaggccgcagaaacgactgatgctccaggaccctctgaaccacgacg

SEQ ID NO: 69 - protein, 77 aa, Conus circumcisus
MFTVFLLVVLATTVVSIPSDRASDGRNAAVNERQPWLVPSTITTCCGYDPGTKCPPCRCNNTCKPKKPKPGKGRRND

SEQ ID NO: 70 - DNA, 234 bp, Conus consors
  CDS (1)..(189)
atgttcaccgtgtttctgttggttgtcttggcaaccactgtcgtttccatcccttcagatcgtgcatctgatggcaggaatgccgcagtccatgagagagcgccttggctggtcccttcgcaaatcacgacttgctgtggttataatccggggacaatgtgcccttcttgcatgtgcactaattcctgctaaaaaaaaatggctgatgctcctggaccctctgaaccacgacgt

SEQ ID NO: 71 - protein, 63 aa, Conus consors
MFTVFLLVVLATTVVSIPSDRASDGRNAAVHERAPWLVPSQITTCCGYNPGTMCPSCMCTNSC

SEQ ID NO: 72 - DNA, 256 bp, Conus consors
  CDS (1)..(225)
atgttcaccgtgtttctgttggttgtcttggcaaccactgtcgtttccatcccttcagatcgtgcatctgatgtcaggaatgccgcagtccacgagagacagaaggatctggtcgttacggccaccacgacttgctgtggttataatccgatgacaatatgccctccttgcatgtgcacttattcctgtccccccaaaaagaaaaaaccaggccgcagaaacgactgatgctccaggaccctctgaaccacgacgt

SEQ ID NO: 73 - protein, 75 aa, Conus consors
MFTVFLLVVLATTVVSIPSDRASDVRNAAVHERQKDLVVTATTTCCGYNPMTICPPCMCTYSCPPKKKKPGRRND

SEQ ID NO: 74 - DNA, 256 bp, Conus magus
  CDS (1)..(225)
atgttcaccgtgtttctgttggttgtcttggcaaccactgtcgtttccatcccttcagatcgtgcatctgatggcaggaatgccgtagtccacgagagagcgcctgagctggtcgttacggccaccacgacttgctgtggttttgatccgatgacatggtgccctccttgcatgtgcacttattcctgttcccaccaaaggaaaaaaccaggccgcagaaacgactgatgctccaggaccctctgaaccacgacgt

SEQ ID NO: 75 - protein, 75 aa, Conus magus
MFTVFLLVVLATTVVSIPSDRASDGRNAVVHERAPELVVTATTTCCGFDPMTWCPPCMCTYSCSHQRKKPGRRND

SEQ ID NO: 76 - DNA, 260 bp, Conus magus
  CDS (1)..(222)
atgttcaccgtgtttctgttggttgtcttggcaaccactgtcgtttccatcccttcagatcgtgcatctgatggcaggaatgccgtagtccacgagagagcgcctgagctggtcgttacggccaccacgaattgctgtggttataatccgatgacaatatgccctccttgcatgtgcacttattcctgtccaccaaaaagaaaaccaggccgcagaaacgactgatgctccaggaccctctgaaccacgacgttcgagca

SEQ ID NO: 77 - protein, 74 aa, Conus magus
MFTVFLLVVLATTVVSIPSDRASDGRNAVVHERAPELVVTATTNCCGYNPMTICPPCMCTYSCPPKRKPGRRND

SEQ ID NO: 78 - DNA, 250 bp, Conus monachus; identical to SEQ ID NO: 58
  CDS (1)..(219)

SEQ ID NO: 79 - protein, 73 aa, Conus monachus; identical to SEQ ID NO: 59

SEQ ID NO: 80 - DNA, 256 bp, Conus monachus
  CDS (1)..(225)
atgttcaccgtgtttccgttggtcgtcttggcaaccactctcgtttccatcccttcagatcgtgcatctgatggcaggaatgccgtagtccacgagagacagccttggctggtcccttcgaaaatcacgaattgctgtggttataatacgatggaaatgtgccctacttgcatgtgcacttattcctgtcgccccaaaaagaaaaaaccaggccgcagaaacgactgatgctccaggaccctctgaaccacgacgt

SEQ ID NO: 81 - protein, 75 aa, Conus monachus
MFTVFPLVVLATTLVSIPSDRASDGRNAVVHERQPWLVPSKITNCCGYNTMEMCPTCMCTYSCRPKKKKPGRRND

SEQ ID NO: 82 - DNA, 256 bp, Conus stercusmuscarum
  CDS (1)..(225)
atgttcaccgtgtttctgttggttgtcttggcaaccactgtcgtttccatcccttcagatcgtgcatctgatggcaggaatgccgcagtcaacgagagagcgccttggctggtcccttcgacaatcacgacttgctgtggatatgatccggggacaatgtgccctccttgcatgtgcaataatacctgtaaaccaacaaaaaaaagaccaggccgcagaaacgactgatgctcccaggaccctctgaaccacgacg

SEQ ID NO: 83 - protein, 75 aa, Conus stercusmuscarum
MFTVFLLVVLATTVVSIPSDRASDGRNAAVNERAPWLVPSTITTCCGYDPGTMCPPCMCNNTCKPTKKRPGRRND

SEQ ID NO: 84 - DNA, 256 bp, Conus stercusmuscarum
  CDS (1)..(225)
atgttcaccgtgtttctgttggttgtcttggcaaccactgtcgtttccatcccttcagatcgtgcatctgatggcaggaatgccgcagtcaacgagagacaaacttggctggtcccttcgacaatcacgacttgctgtggatatgatccggggacaatgtgccctacttgcatgtgcgataatacctgtaaaccaaaacccaaaaaatcaggccgcagaaacgactgatgctccaggaccctctgaaccacgacgt

SEQ ID NO: 85 - protein, 75 aa, Conus stercusmuscarum
MFTVFLLVVLATTVVSIPSDRASDGRNAAVNERQTWLVPSTITTCCGYDPGTMCPTCMCDNTCKPKPKKSGRRND

SEQ ID NO: 86 - DNA, 263 bp, Conus stercusmuscarum
  CDS (1)..(225)
atgttcaccgtgtttctgttggttgtcttggcaaccactgtcgtttccatcccttcagatcgtgcatctgatggcaggaatgccgaagtcaacgagagagcgccttggctggtcccttcgacaatcacgacttgctgtggatatgatccggggtcaatgtgccctccttgcatgtgcaataatacctgtaaaccaaaacccaaaaaatcaggccgcagaaaccactgatgctccaggaccctctgaaccacgacgttcgagca

SEQ ID NO: 87 - protein, 75 aa, Conus stercusmuscarum
MFTVFLLVVLATTVVSIPSDRASDGRNAEVNERAPWLVPSTITTCCGYDPGSMCPPCMCNNTCKPKPKKSGRRNH

SEQ ID NO: 88 - DNA, 263 bp, Conus striatus
  CDS (1)..(192)
atgttcaccgtgtttctgttggttgtcttggcaaccaatgtcgtttccaccccttcagatcgtgcatctgatggcaggaatgccgcagtccacgagagacagaagagtctggtcccttcggtcatcacgacttgctgtggatatgatccggggacaatgtgccctccttgcaggtgcactaatagctgtggttaaccaaaacccaaaacaggccgcagaaacgactgatgctccaggaccctctgaaccacgacgttcgagca

SEQ ID NO: 89 - protein, 64 aa, Conus striatus
MFTVFLLVVLATNVVSTPSDRASDGRNAAVHERQKSLVPSVITTCCGYDPGTMCPPCRCTNSCG

SEQ ID NO: 90 - DNA, 263 bp, Conus striatus
  CDS (1)..(225)
atgttcaccgtgtttctgtcggttgtcttggcaaccactgtcgtttccaccccttcagatcgtgcatctgatggcaggaatgccgcagtccacgagagacagaaggagctggtcccttcggtcatcacgacttgctgtggatatgatccggggacaatgtgccctccttgcaggtgcactaattcctgtccaacaaaaccgaaaaaaccaggccgcagaaacgactgatgctccaggaccctctgaaccacgacgttcgagca

SEQ ID NO: 91 - protein, 75 aa, Conus striatus
MFTVFLSVVLATTVVSTPSDRASDGRNAAVHERQKELVPSVITTCCGYDPGTMCPPCRCTNSCPTKPKKPGRRND

SEQ ID NO: 92 - DNA, 232 bp, Conus striolatus
  CDS (1)..(189)
atgttcaccgtgtttctgttggttgtcttggcaaccactgtcgtttccatcccttcagatcgtgcatatgatggcaagaatgccgcagtccacgagagacaatcttggctggtcccttcgacaatcacgacttgctgtggttatagtccggggacaatgtgccctccttgcatgtgcactaatacctgctaaaaaaatggctgatgctccaggaccctctgaaccacgacgt

SEQ ID NO: 93 - protein, 63 aa, Conus striolatus
MFTVFLLVVLATTVVSIPSDRAYDGKNAAVHERQSWLVPSTITTCCGYSPGTMCPPCMCTNTC

SEQ ID NO: 94 - DNA, 256 bp, Conus sulcatus
  CDS (1)..(225)
atgttcaccgtgtttctgttggttgtcttggcaaccactgtcgtttccatcccttcagatcgtgcatctgatgtcaggaatgccgcagtccacgagagacagaaggatctggtcgttacggccaccacgacttgctgtggttataatccgatgacaatgtgccctccttgcatgcgcacttattcctgttcccccaaaaagaaaaaaccaggccgcagaaacgactgatgctccaggaccctctgaaccacgacgt

SEQ ID NO: 95 - protein, 75 aa, Conus sulcatus
MFTVFLLVVLATTVVSIPSDRASDVRNAAVHERQKDLVVTATTTCCGYNPMTMCPPCMRTYSCSPKKKKPGRRND

SEQ ID NO: 96 - DNA, 256 bp, Conus sulcatus
  CDS (1)..(225)
atgttcaccgtgtttctgttggttgtcttggcaaccactgtcgtttccaccccttcagatcgtgcatctgatggcaggaatgccgcagtccacgggagacagaaggagctggtcccttcggtcatcacgacttgctgtggatatgatccggggacaatgtgccctccttgcaggtgcactaattcctgtccaacaaaaccgaaaaaaccaggccgcagaaacgactgatgctccaggaccctctgaaccacgacgt

SEQ ID NO: 97 - protein, 75 aa, Conus sulcatus
MFTVFLLVVLATTVVSTPSDRASDGRNAAVHGRQKELVPSVITTCCGYDPGTMCPPCRCTNSCPTKPKKPGRRND